SEQUENCE LISTING (161 sequences)

SEQ ID NO: 1 - DNA, 44 bp, Artificial Sequence
  Description of Artificial Sequence Synthetic primer
tagcgtttaaacttaagcttatggtgagcaagggcgaggagctg

SEQ ID NO: 2 - DNA, 47 bp, Artificial Sequence
  Description of Artificial Sequence Synthetic primer
tcggtcatggtaccaagcttcttgtacagctcgtccatgccgagagt

SEQ ID NO: 3 - DNA, 40 bp, Artificial Sequence
  Description of Artificial Sequence Synthetic primer
gtggttagccagcgtttccctcagaacagcatcggtgcag

SEQ ID NO: 4 - DNA, 36 bp, Artificial Sequence
  Description of Artificial Sequence Synthetic primer
tcaagtgtatcatatggtggttagccagcgtttccc

SEQ ID NO: 5 - DNA, 36 bp, Artificial Sequence
  Description of Artificial Sequence Synthetic primer
gcgtacttggcatatgctgcaccgatgctgttctga

SEQ ID NO: 6 - DNA, 42 bp, Artificial Sequence
  Description of Artificial Sequence Synthetic primer
gaccggcgcgccggatccatggctatcgacgaaaacaaacag

SEQ ID NO: 7 - DNA, 46 bp, Artificial Sequence
  Description of Artificial Sequence Synthetic primer
cactggactagtggatccttaaaaatcttcgttagtttctgctacg

SEQ ID NO: 8 - DNA, 36 bp, Artificial Sequence
  Description of Artificial Sequence Synthetic primer
gcaggaaagaacatgtgtggttagccagcgtttccc

SEQ ID NO: 9 - DNA, 36 bp, Artificial Sequence
  Description of Artificial Sequence Synthetic primer
ccttttgctcacatgtctgcaccgatgctgttctga

SEQ ID NO: 10 - DNA, 22 bp, Artificial Sequence
  Description of Artificial Sequence Synthetic primer
gcagagctctctggctaactag

SEQ ID NO: 11 - DNA, 20 bp, Artificial Sequence
  Description of Artificial Sequence Synthetic primer
gacgcaccctgtctgactac

SEQ ID NO: 12 - DNA, 25 bp, Artificial Sequence
  Description of Artificial Sequence Synthetic primer
aatgacgtatgttcccatagtaacg

SEQ ID NO: 13 - DNA, 20 bp, Artificial Sequence
  Description of Artificial Sequence Synthetic primer
gcttcctcgctcactgactc

SEQ ID NO: 14 - DNA, 25 bp, Artificial Sequence
  Description of Artificial Sequence Synthetic primer
atacgtagatgtactgccaagtagg

SEQ ID NO: 15 - DNA, 19 bp, Artificial Sequence
  Description of Artificial Sequence Synthetic primer
cctctgacttgagcgtcga

SEQ ID NO: 16 - DNA, 40 bp, Artificial Sequence
  Description of Artificial Sequence Synthetic primer
tattaaactctcacctcccatgttgctcaaagaaccatat

SEQ ID NO: 17 - DNA, 36 bp, Artificial Sequence
  Description of Artificial Sequence Synthetic primer
tcaagtgtatcatatgtattaaactctcacctccca

SEQ ID NO: 18 - DNA, 36 bp, Artificial Sequence
  Description of Artificial Sequence Synthetic primer
gcgtacttggcatatgatatggttctttgagcaaca

SEQ ID NO: 19 - DNA, 40 bp, Artificial Sequence
  Description of Artificial Sequence Synthetic primer
caagatgcgctagtggacagattgctgaccaggggcttga

SEQ ID NO: 20 - DNA, 36 bp, Artificial Sequence
  Description of Artificial Sequence Synthetic primer
tcaagtgtatcatatgcaagatgcgctagtggacag

SEQ ID NO: 21 - DNA, 37 bp, Artificial Sequence
  Description of Artificial Sequence Synthetic primer
gcgtacttggcatatgtcaagcccctggtcagcaatc

SEQ ID NO: 22 - DNA, 40 bp, Artificial Sequence
  Description of Artificial Sequence Synthetic primer
agcctgtggagcgattaaaccgtgcgcggagctgcttctt

SEQ ID NO: 23 - DNA, 37 bp, Artificial Sequence
  Description of Artificial Sequence Synthetic primer
tcaagtgtatcatatgagcctgtggagcgattaaacc

SEQ ID NO: 24 - DNA, 35 bp, Artificial Sequence
  Description of Artificial Sequence Synthetic primer
gcgtacttggcatatgaagaagcagctccgcgcac

SEQ ID NO: 25 - DNA, 40 bp, Artificial Sequence
  Description of Artificial Sequence Synthetic primer
cacttttcgacatagtgtggtggtgccctatgagccgcct

SEQ ID NO: 26 - DNA, 38 bp, Artificial Sequence
  Description of Artificial Sequence Synthetic primer
tgcattagttattaatcacttttcgacatagtgtggtg

SEQ ID NO: 27 - DNA, 34 bp, Artificial Sequence
  Description of Artificial Sequence Synthetic primer
attgattactattaataggcggctcatagggcac

SEQ ID NO: 28 - DNA, 23 bp, Artificial Sequence
  Description of Artificial Sequence Synthetic primer
gcgttactatgggaacatacgtc

SEQ ID NO: 29 - DNA, 21 bp, Artificial Sequence
  Description of Artificial Sequence Synthetic primer
ctgcgttatcccctgattctg

SEQ ID NO: 30 - DNA, 25 bp, Artificial Sequence
  Description of Artificial Sequence Synthetic primer
ctaaagaaggttgcgcagttagcag

SEQ ID NO: 31 - DNA, 25 bp, Artificial Sequence
  Description of Artificial Sequence Synthetic primer
gctgctttacgtttggtgctttcag

SEQ ID NO: 32 - DNA, 23 bp, Artificial Sequence
  Description of Artificial Sequence Synthetic primer
taaggactcgcctctgcacaaag

SEQ ID NO: 33 - DNA, 24 bp, Artificial Sequence
  Description of Artificial Sequence Synthetic primer
acttcctctggactcttgtcattg

SEQ ID NO: 34 - DNA, 21 bp, Artificial Sequence
  Description of Artificial Sequence Synthetic primer
cagcttgccgtaggtggcatc

SEQ ID NO: 35 - DNA, 18 bp, Artificial Sequence
  Description of Artificial Sequence Synthetic primer
gtcaccgccgacgtcgag

SEQ ID NO: 36 - DNA, 21 bp, Artificial Sequence
  Description of Artificial Sequence Synthetic primer
ccggtgccggatctatgcaga

SEQ ID NO: 37 - DNA, 24 bp, Artificial Sequence
  Description of Artificial Sequence Synthetic primer
gctgaacttgtggccgtttacgtc

SEQ ID NO: 38 - DNA, 21 bp, Artificial Sequence
  Description of Artificial Sequence Synthetic primer
ggaggacctgcctctcgtcag

SEQ ID NO: 39 - DNA, 26 bp, Artificial Sequence
  Description of Artificial Sequence Synthetic primer
acctcccaatactccatccacagatg

SEQ ID NO: 40 - DNA, 24 bp, Artificial Sequence
  Description of Artificial Sequence Synthetic primer
cgcttcgagatgttccgagagctg

SEQ ID NO: 41 - DNA, 25 bp, Artificial Sequence
  Description of Artificial Sequence Synthetic primer
gtcaggcccttctgtcttgaacatg

SEQ ID NO: 42 - DNA, 24 bp, Artificial Sequence
  Description of Artificial Sequence Synthetic primer
aaggtgccacagacacagacttag

SEQ ID NO: 43 - DNA, 23 bp, Artificial Sequence
  Description of Artificial Sequence Synthetic primer
tttcacatgggtgtccatgactg

SEQ ID NO: 44 - DNA, 24 bp, Artificial Sequence
  Description of Artificial Sequence Synthetic primer
tctaagtgtgggaaaacatttaca

SEQ ID NO: 45 - DNA, 25 bp, Artificial Sequence
  Description of Artificial Sequence Synthetic primer
tccacgtttactcttcttcgtttct

SEQ ID NO: 46 - DNA, 27 bp, Artificial Sequence
  Description of Artificial Sequence Synthetic primer
caccaacgggaaacgctggctaaccac

SEQ ID NO: 47 - DNA, 27 bp, Artificial Sequence
  Description of Artificial Sequence Synthetic primer
gttggtgtcagaacagcatcggtgcag

SEQ ID NO: 48 - DNA, 27 bp, Artificial Sequence
  Description of Artificial Sequence Synthetic primer
tcaatctgtttaatcgctccacaggct

SEQ ID NO: 49 - DNA, 27 bp, Artificial Sequence
  Description of Artificial Sequence Synthetic primer
agattgacgtgcgcggagctgcttctt

SEQ ID NO: 50 - DNA, 27 bp, Artificial Sequence
  Description of Artificial Sequence Synthetic primer
caccaactgggaggtgagagtttaata

SEQ ID NO: 51 - DNA, 27 bp, Artificial Sequence
  Description of Artificial Sequence Synthetic primer
gttggtgtgttgctcaaagaaccatat

SEQ ID NO: 52 - DNA, 27 bp, Artificial Sequence
  Description of Artificial Sequence Synthetic primer
caccaacctgtccactagcgcatcttg

SEQ ID NO: 53 - DNA, 27 bp, Artificial Sequence
  Description of Artificial Sequence Synthetic primer
gttggtgattgctgaccaggggcttga

SEQ ID NO: 54 - DNA, 27 bp, Artificial Sequence
  Description of Artificial Sequence Synthetic primer
caccaacccacactatgtcgaaaagag

SEQ ID NO: 55 - DNA, 27 bp, Artificial Sequence
  Description of Artificial Sequence Synthetic primer
gttggtgtggtgccctatgagccgcct

SEQ ID NO: 56 - DNA, 26 bp, Artificial Sequence
  Description of Artificial Sequence Synthetic primer
caggtcatcatcatctggcaatgagc

SEQ ID NO: 57 - DNA, 26 bp, Artificial Sequence
  Description of Artificial Sequence Synthetic primer
cggatgtcacacgtcacacttcatga

SEQ ID NO: 58 - DNA, 25 bp, Artificial Sequence
  Description of Artificial Sequence Synthetic primer
cgaaaggcaaggaggaagcttatct

SEQ ID NO: 59 - DNA, 26 bp, Artificial Sequence
  Description of Artificial Sequence Synthetic primer
cgaatttcagttctgtacatctgcct

SEQ ID NO: 60 - DNA, 25 bp, Artificial Sequence
  Description of Artificial Sequence Synthetic primer
gatactttgtaggagctgagcacag

SEQ ID NO: 61 - DNA, 25 bp, Artificial Sequence
  Description of Artificial Sequence Synthetic primer
ctgagtgaaaccttgttctaagctg

SEQ ID NO: 62 - DNA, 24 bp, Artificial Sequence
  Description of Artificial Sequence Synthetic primer
cccagaggagttagatgacgtcac

SEQ ID NO: 63 - DNA, 24 bp, Artificial Sequence
  Description of Artificial Sequence Synthetic primer
cacagtccgagacgccgccatgac

SEQ ID NO: 64 - DNA, 23 bp, Artificial Sequence
  Description of Artificial Sequence Synthetic primer
cacaggccggtggaatgggtcca

SEQ ID NO: 65 - DNA, 24 bp, Artificial Sequence
  Description of Artificial Sequence Synthetic primer
ctacctcccctcacctactctgtc

SEQ ID NO: 66 - DNA, 24 bp, Artificial Sequence
  Description of Artificial Sequence Synthetic primer
ccttgtccctgaccagcctccgac

SEQ ID NO: 67 - DNA, 26 bp, Artificial Sequence
  Description of Artificial Sequence Synthetic primer
ctaaaggcgcccccaggtcactcatc

SEQ ID NO: 68 - DNA, 25 bp, Artificial Sequence
  Description of Artificial Sequence Synthetic primer
agggacaggaggtatcggaaggctc

SEQ ID NO: 69 - DNA, 25 bp, Artificial Sequence
  Description of Artificial Sequence Synthetic primer
ccttttaacagtttaagctgaccac

SEQ ID NO: 70 - DNA, 25 bp, Artificial Sequence
  Description of Artificial Sequence Synthetic primer
gaaaatgagtagaatgtacacagag

SEQ ID NO: 71 - DNA, 26 bp, Artificial Sequence
  Description of Artificial Sequence Synthetic primer
gtgaacaatggttaatttgtttctag

SEQ ID NO: 72 - DNA, 26 bp, Artificial Sequence
  Description of Artificial Sequence Synthetic primer
ctaaatagctgaagcttctggcttga

SEQ ID NO: 73 - DNA, 24 bp, Artificial Sequence
  Description of Artificial Sequence Synthetic primer
tggcaaagattctatgcgctagtt

SEQ ID NO: 74 - DNA, 25 bp, Artificial Sequence
  Description of Artificial Sequence Synthetic primer
catagagcactttcaagcatggact

SEQ ID NO: 75 - DNA, 24 bp, Artificial Sequence
  Description of Artificial Sequence Synthetic primer
cagccactttcaccaagtacactg

SEQ ID NO: 76 - DNA, 24 bp, Artificial Sequence
  Description of Artificial Sequence Synthetic primer
ggcactaaaaactagacatcagag

SEQ ID NO: 77 - DNA, 27 bp, Artificial Sequence
  Description of Artificial Sequence Synthetic primer
ctgatcctagtgaaatattttttaaac

SEQ ID NO: 78 - DNA, 25 bp, Artificial Sequence
  Description of Artificial Sequence Synthetic primer
gtatgacccattgtaactacatcag

SEQ ID NO: 79 - DNA, 25 bp, Artificial Sequence
  Description of Artificial Sequence Synthetic primer
gataaaaccaacaggtgaagacatg

SEQ ID NO: 80 - DNA, 24 bp, Artificial Sequence
  Description of Artificial Sequence Synthetic primer
ccagttctagaatcatagctctga

SEQ ID NO: 81 - DNA, 27 bp, Artificial Sequence
  Description of Artificial Sequence Synthetic primer
aactgtttcccttttgcttcttgatag

SEQ ID NO: 82 - DNA, 23 bp, Artificial Sequence
  Description of Artificial Sequence Synthetic primer
cggcttcagttgcttagagacgt

SEQ ID NO: 83 - DNA, 28 bp, Artificial Sequence
  Description of Artificial Sequence Synthetic primer
agatacatctttaaaaatagcaatggtg

SEQ ID NO: 84 - DNA, 25 bp, Artificial Sequence
  Description of Artificial Sequence Synthetic primer
gtcaattgaaggatacacagagaag

SEQ ID NO: 85 - DNA, 24 bp, Artificial Sequence
  Description of Artificial Sequence Synthetic primer
ttcattacaagtagatcacacact

SEQ ID NO: 86 - DNA, 26 bp, Artificial Sequence
  Description of Artificial Sequence Synthetic primer
gaaatagtagacatgattgggtctca

SEQ ID NO: 87 - DNA, 26 bp, Artificial Sequence
  Description of Artificial Sequence Synthetic primer
gcaacaaaaacaatgttggcttgtac

SEQ ID NO: 88 - DNA, 26 bp, Artificial Sequence
  Description of Artificial Sequence Synthetic primer
cacttcagcctatttgaccttcactg

SEQ ID NO: 89 - DNA, 24 bp, Artificial Sequence
  Description of Artificial Sequence Synthetic primer
aggaacacagagaatgtttcaatg

SEQ ID NO: 90 - DNA, 25 bp, Artificial Sequence
  Description of Artificial Sequence Synthetic primer
tttcaaggccttaggaaacgctgac

SEQ ID NO: 91 - DNA, 23 bp, Artificial Sequence
  Description of Artificial Sequence Synthetic primer
aagaaacactgtcatctctggga

SEQ ID NO: 92 - DNA, 25 bp, Artificial Sequence
  Description of Artificial Sequence Synthetic primer
aatgcttcagccttctaattctcag

SEQ ID NO: 93 - DNA, 27 bp, Artificial Sequence
  Description of Artificial Sequence Synthetic primer
gcattagtataacactgaacaagagtg

SEQ ID NO: 94 - DNA, 25 bp, Artificial Sequence
  Description of Artificial Sequence Synthetic primer
acagagtctcatctccgcaggtgca

SEQ ID NO: 95 - DNA, 25 bp, Artificial Sequence
  Description of Artificial Sequence Synthetic primer
tctgcatcgaagcctgactgaagtc

SEQ ID NO: 96 - DNA, 24 bp, Artificial Sequence
  Description of Artificial Sequence Synthetic primer
tgctgggtgacattggatgctgag

SEQ ID NO: 97 - DNA, 25 bp, Artificial Sequence
  Description of Artificial Sequence Synthetic primer
ggttgaactttctccaaccatgcag

SEQ ID NO: 98 - DNA, 24 bp, Artificial Sequence
  Description of Artificial Sequence Synthetic primer
gtcaaccttctcaggtggctctga

SEQ ID NO: 99 - DNA, 24 bp, Artificial Sequence
  Description of Artificial Sequence Synthetic primer
cctaaggaggttgaaccatctgca

SEQ ID NO: 100 - DNA, 27 bp, Artificial Sequence
  Description of Artificial Sequence Synthetic primer
taattttcaggtctatttagctgctga

SEQ ID NO: 101 - DNA, 24 bp, Artificial Sequence
  Description of Artificial Sequence Synthetic primer
ggtgagtcttctccaatccagcag

SEQ ID NO: 102 - DNA, 25 bp, Artificial Sequence
  Description of Artificial Sequence Synthetic primer
aagattcaacctctccagaagactc

SEQ ID NO: 103 - DNA, 25 bp, Artificial Sequence
  Description of Artificial Sequence Synthetic primer
tccagaggaggtggcaccttctgca

SEQ ID NO: 104 - DNA, 25 bp, Artificial Sequence
  Description of Artificial Sequence Synthetic primer
ctggatgtcttcaagggtctctgga

SEQ ID NO: 105 - DNA, 26 bp, Artificial Sequence
  Description of Artificial Sequence Synthetic primer
actgtattccggcagcctgcctccag

SEQ ID NO: 106 - DNA, 26 bp, Artificial Sequence
  Description of Artificial Sequence Synthetic primer
gaatatactcacagtttctcggtcca

SEQ ID NO: 107 - DNA, 26 bp, Artificial Sequence
  Description of Artificial Sequence Synthetic primer
ttcttcacagaaatttccaaggaaac

SEQ ID NO: 108 - DNA, 25 bp, Artificial Sequence
  Description of Artificial Sequence Synthetic primer
gattaactaacttacaaatactgga

SEQ ID NO: 109 - DNA, 30 bp, Artificial Sequence
  Description of Artificial Sequence Synthetic primer
taactttttcattttccagaattctcagtg

SEQ ID NO: 110 - DNA, 24 bp, Artificial Sequence
  Description of Artificial Sequence Synthetic primer
cttctatttcacttacaagttgtg

SEQ ID NO: 111 - DNA, 29 bp, Artificial Sequence
  Description of Artificial Sequence Synthetic primer
tgtctttttttgtagaaatctgggctgca

SEQ ID NO: 112 - DNA, 24 bp, Artificial Sequence
  Description of Artificial Sequence Synthetic primer
tgcatggcaccaagaatgttacca

SEQ ID NO: 113 - DNA, 25 bp, Artificial Sequence
  Description of Artificial Sequence Synthetic primer
gactccagactatatctccacagtc

SEQ ID NO: 114 - DNA, 25 bp, Artificial Sequence
  Description of Artificial Sequence Synthetic primer
actgggggctcttgcgtagcacatg

SEQ ID NO: 115 - DNA, 25 bp, Artificial Sequence
  Description of Artificial Sequence Synthetic primer
ggctgtgatgtgtgagcctgcactg

SEQ ID NO: 116 - DNA, 25 bp, Artificial Sequence
  Description of Artificial Sequence Synthetic primer
ggaattggttgtagttgtggaatcg

SEQ ID NO: 117 - DNA, 24 bp, Artificial Sequence
  Description of Artificial Sequence Synthetic primer
gatgacccccgtgaaccctgcgac

SEQ ID NO: 118 - DNA, 25 bp, Artificial Sequence
  Description of Artificial Sequence Synthetic primer
ctaaagaaggttgcgcagttagcag

SEQ ID NO: 119 - DNA, 25 bp, Artificial Sequence
  Description of Artificial Sequence Synthetic primer
catatcagtctgtgggatctggtac

SEQ ID NO: 120 - DNA, 26 bp, Artificial Sequence
  Description of Artificial Sequence Synthetic primer
ccatatcagtctgtgggatctggtat

SEQ ID NO: 121 - DNA, 24 bp, Artificial Sequence
  Description of Artificial Sequence Synthetic primer
acttcctctggactcttgtcattg

SEQ ID NO: 122 - DNA, 25 bp, Artificial Sequence
  Description of Artificial Sequence Synthetic primer
ggtcaacttgtattcagcaggtacc

SEQ ID NO: 123 - DNA, 25 bp, Artificial Sequence
  Description of Artificial Sequence Synthetic primer
ggtcaacttgtattcagcaggtacg

SEQ ID NO: 124 - DNA, 24 bp, Artificial Sequence
  Description of Artificial Sequence Synthetic primer
ttgccatctttagacatgcacatg

SEQ ID NO: 125 - DNA, 29 bp, Artificial Sequence
  Description of Artificial Sequence Synthetic primer
acataaagttcattgttactagcatcatc

SEQ ID NO: 126 - DNA, 26 bp, Artificial Sequence
  Description of Artificial Sequence Synthetic primer
gaatacagtgttcatctggtttcatc

SEQ ID NO: 127 - DNA, 46 bp, Artificial Sequence
  Description of Artificial Sequence Synthetic primer
tctggcctcggcggccaagcttttgttgcattggatatccttggtc

SEQ ID NO: 128 - DNA, 47 bp, Artificial Sequence
  Description of Artificial Sequence Synthetic primer
ataccctctagtgtctaagcttctaaattatgcctgagactgcctag

SEQ ID NO: 129 - DNA, 20 bp, Artificial Sequence
  Description of Artificial Sequence Synthetic oligonucleotide
attaaatccagatggcgcgc

SEQ ID NO: 130 - DNA, 19 bp, Artificial Sequence
  Description of Artificial Sequence Synthetic oligonucleotide
ttaaatccagatggcgcgc

SEQ ID NO: 131 - DNA, 20 bp, Artificial Sequence
  Description of Artificial Sequence Synthetic oligonucleotide
ttaaatccagatggcgcgcc

SEQ ID NO: 132 - DNA, 20 bp, Artificial Sequence
  Description of Artificial Sequence Synthetic oligonucleotide
ttaaatccagatggcgcgcc

SEQ ID NO: 133 - DNA, 20 bp, Artificial Sequence
  Description of Artificial Sequence Synthetic oligonucleotide
ggaaacagttgagggtcctc

SEQ ID NO: 134 - DNA, 19 bp, Artificial Sequence
  Description of Artificial Sequence Synthetic oligonucleotide
gaaacagttgagggtcctc

SEQ ID NO: 135 - DNA, 20 bp, Artificial Sequence
  Description of Artificial Sequence Synthetic oligonucleotide
gaaacagttgagggtcctca

SEQ ID NO: 136 - DNA, 20 bp, Artificial Sequence
  Description of Artificial Sequence Synthetic oligonucleotide
tagccatgagatctaggtct

SEQ ID NO: 137 - DNA, 20 bp, Artificial Sequence
  Description of Artificial Sequence Synthetic oligonucleotide
tccacaaatagatggcatct

SEQ ID NO: 138 - DNA, 19 bp, Artificial Sequence
  Description of Artificial Sequence Synthetic oligonucleotide
tttcccagagatgacagtg

SEQ ID NO: 139 - DNA, 20 bp, Artificial Sequence
  Description of Artificial Sequence Synthetic oligonucleotide
tcccgagtagctgggactac

SEQ ID NO: 140 - DNA, 20 bp, Artificial Sequence
  Description of Artificial Sequence Synthetic oligonucleotide
acaccaccacgcctggctaa

SEQ ID NO: 141 - DNA, 20 bp, Artificial Sequence
  Description of Artificial Sequence Synthetic oligonucleotide
tctacctcctgagggaaccc

SEQ ID NO: 142 - DNA, 19 bp, Artificial Sequence
  Description of Artificial Sequence Synthetic oligonucleotide
gacccagcagatggcccca

SEQ ID NO: 143 - DNA, 20 bp, Artificial Sequence
  Description of Artificial Sequence Synthetic oligonucleotide
gacccagcagatggccccat

SEQ ID NO: 144 - DNA, 20 bp, Artificial Sequence
  Description of Artificial Sequence Synthetic oligonucleotide
taacctgaaggtgaaactgg

SEQ ID NO: 145 - DNA, 20 bp, Artificial Sequence
  Description of Artificial Sequence Synthetic oligonucleotide
atgggctgggggtggtggct

SEQ ID NO: 146 - DNA, 19 bp, Artificial Sequence
  Description of Artificial Sequence Synthetic oligonucleotide
gtttcaacagagggcaaag

SEQ ID NO: 147 - DNA, 20 bp, Artificial Sequence
  Description of Artificial Sequence Synthetic oligonucleotide
gtttcaacagagggcaaagc

SEQ ID NO: 148 - DNA, 20 bp, Artificial Sequence
  Description of Artificial Sequence Synthetic oligonucleotide
tgggctgggggtggtggctc

SEQ ID NO: 149 - DNA, 20 bp, Artificial Sequence
  Description of Artificial Sequence Synthetic oligonucleotide
ctgcccaccagatgccagga

SEQ ID NO: 150 - DNA, 19 bp, Artificial Sequence
  Description of Artificial Sequence Synthetic oligonucleotide
tgcccaccagatgccagga

SEQ ID NO: 151 - DNA, 20 bp, Artificial Sequence
  Description of Artificial Sequence Synthetic oligonucleotide
ttcccaggaggtgctggtct

SEQ ID NO: 152 - DNA, 116 bp, Homo sapiens
atgaatgcaaagaaacttaatttcaaggccttaggaaacgctgactgtcttcattcttgcttcttgtttgaaggtaatgtgagtggtttcttttcccagagatgacagtgtttctt

SEQ ID NO: 153 - DNA, 27 bp, Artificial Sequence
  Description of Artificial Sequence Synthetic oligonucleotide
tcaatctgaagacagtcagcgtttcct

SEQ ID NO: 154 - DNA, 27 bp, Artificial Sequence
  Description of Artificial Sequence Synthetic oligonucleotide
agattgaattcttgcttcttgtttgaa

SEQ ID NO: 155 - DNA, 27 bp, Artificial Sequence
  Description of Artificial Sequence Synthetic oligonucleotide
caccaacgggaaacgctggctaaccac

SEQ ID NO: 156 - DNA, 27 bp, Artificial Sequence
  Description of Artificial Sequence Synthetic oligonucleotide
gttggtgtcagaacagcatcggtgcag

SEQ ID NO: 157 - DNA, 20 bp, Homo sapiens
ttttcccagagatgacagtg

SEQ ID NO: 158 - DNA, 40 bp, Homo sapiens
aggaaacgctgactgtcttcattcttgcttcttgtttgaa

SEQ ID NO: 159 - DNA, 40 bp, Homo sapiens
ttcaaacaagaagcaagaatgaagacagtcagcgtttcct

SEQ ID NO: 160 - DNA, 40 bp, Homo sapiens; identical to SEQ ID NO: 3

SEQ ID NO: 161 - DNA, 40 bp, Homo sapiens
ctgcaccgatgctgttctgagggaaacgctggctaaccac

(19) United States
(12) Patent Application Publication
(10) Pub. No.: US 2013/0273556 A1
(43) Pub. Date: Oct. 17, 2013

(54) MODULATION OF GENE EXPRESSION BY LOCKED NUCLEIC ACIDS

(71) Applicant: The Board of Trustees of the Leland Stanford Junior University, Palo Alto, CA (US)

(72) Inventors: Andrew R. Hoffman, Palo Alto, CA (US); Jianqun Ling, Nanjing (CN)

(73) Assignee: US GOVERNMENT, DEPARTMENT OF VETERANS AFFAIRS, Washington, DC (US)

(21) Appl. No.: 13/882,845

(22) PCT Filed: Nov. 4, 2011
(86) PCT No.: PCT/US11/59421

§ 371 Date: Jun. 26, 2013

Related U.S. Application Data
(60) Provisional application No. 61/411,397, filed on Nov. 8, 2010.

(51) Int. Cl. C12N 15/90; C12N 15/113; C12N 15/85

(52) U.S. Cl. CPC C12N 15/907; C12N 15/85; C12N 15/113
USPC 435/6.19; 435/462; 435/320.1; 435/375; 435/366

(57) ABSTRACT

Disclosed are methods for suppressing or altering gene expression by locked nucleic acids that have high binding affinity for target sequences in genomic DNA. The present methods include a step of recombination to insert a foreign DNA fragment into a specific target site in a genome with greater efficiency than with current techniques. Using a protein to anchor a locked nucleic acid targeting construct at a specific site, DNA recombination between that site and foreign DNA is induced by employing the cell's innate repair machinery. As a result of this recombination, gene transcription can be effectively suppressed. The present methods work in all mammalian cells, and provide a rapid methodology to examine gene function. The foreign DNA can carry markers for ease of screening. The technique is applied in the production of stable cell lines with inserted or deleted genes.

### CROSS-REFERENCE TO RELATED APPLICATIONS

This application claims priority from U.S. Provisional Patent Application No. 61/411,397 filed on Nov. 8, 2010, which is hereby incorporated by reference in its entirety.

### STATEMENT OF GOVERNMENTAL SUPPORT

This invention was made with government support under Grant No. NF050184 awarded by the Department of Defense and by the Research Service of Department of Veterans Affairs. The government has certain rights in this invention.

### REFERENCE TO SEQUENCE LISTING, COMPUTER PROGRAM, OR COMPACT DISK

Applicants assert that the written copy of the Sequence Listing is identical to the Sequence Listing in computer readable form found on the accompanying computer file. Applicants incorporate the contents of the sequence listing by reference in its entirety.

### BACKGROUND OF THE INVENTION

1. Field of the Invention

The present invention relates to the field of nucleic acids, and, more particularly to nucleic acid constructs used to modify expression of a gene by interacting with sequences involved in expression of that gene and to constructs employing modified, or “locked”, nucleic acids.

2. Related Art

Presented below is background information on certain aspects of the present invention as they may relate to technical features referred to in the detailed description, but not necessarily described in detail. That is, individual parts or methods used in the present invention may be described in greater detail in the materials discussed below, which materials may provide further guidance to those skilled in the art for making or using certain aspects of the present invention as claimed. The discussion below should not be construed as an admission as to the relevance of the information to any claims herein or the prior art effect of the material described.

Modulation of gene expression may help discern the function of genes, characterize biological pathways and promote our understanding of molecular mechanisms underlying disease states. Conventionally, gene expression can be manipulated at the RNA level, using antisense RNA, short hairpin RNA (shRNA), small interfering RNA (siRNA), microRNA (miRNA), and peptide nucleic acid (PNA) to regulate gene expression, or at the DNA level, using homologous recombination to knock out or insert a gene into a genome. These techniques suffer from very low efficiency (10⁻⁶) in mammalian somatic cells and may take 4-6 months to complete. The recently developed Zinc Finger Nuclease (ZFN) technology provides a more efficient way (1-20%) to manipulate a target gene region, but it also takes several months to screen the zinc finger protein library to find an ideal combination and make constructs for expressing recombinant zinc finger nucleases.

There are a number of traditional molecular tools that can be used to manipulate specific gene expression in vivo, including gene knockout using Cre-LoxP recombinant technology and zinc finger nuclease (ZFN) technology, but these techniques are time consuming and suffer from low efficiency. Moreover, these tools change the DNA content of the target region.

Recently, Ge et al (“Zorro locked nucleic acid induces sequence-specific gene silencing”, Faseb J, 2007. 21(8): p. 1902-14) generated a novel sequence-specific anti-gene reagent composed of two locked nucleic acid (LNA) oligonucleotides with a Z-like shape, and named the construct “Zorro LNA”. They showed that Zorro LNA was able to invade target duplex DNA strands more effectively than a standard LNA oligonucleotide. They microinjected a Zorro LNA targeting the DNA fragment between the CMV promoter and the d2EGFP coding region into NIH-3T3 cells that stably expressed d2EGFP. d2EGFP expression was inhibited by Zorro LNA in the majority of the microinjected cells, but only if the LNA binding sites were located in the intron, and not if the binding sites were located upstream of the gene. However, long-range DNA interactions play a vital role in the regulation of gene transcription.

The invention, as described below, involves two aspects of gene modulation using locked nucleic acids in a linked LNA (e.g. “Zorro”) configuration. The first aspect has to do with a novel DNA recombination technology to regulate gene expression with a high degree of efficiency and specificity. The second aspect has to do with an effective and convenient technique to alter DNA long-range interactions and thereby manipulate gene expression without altering total DNA content or linear sequence.

### SPECIFIC PATENTS AND PUBLICATIONS

Ge, R., et al., “Zorro locked nucleic acid induces sequence-specific gene silencing”, Faseb J, 2007. 21(8): p. 1902-14.
US 2010/0041738 by Smith et al. (including Ge), published Feb. 18, 2010, entitled “Hybridization-stabilizing construct”.
US 2010/0186124 by Bundock et al., published Jul. 22, 2010, entitled “Targeted nucleotide exchange with LNA modified oligonucleotides”.
U.S. Pat. No. 5,527,899 to Froehler, issued Jun. 18, 1996, entitled “Oligonucleotides with inverted polarity”.
U.S. Pat. No. 6,268,490 to Imanishi et al., entitled “Bicyclonucleoside and oligonucleotide analogues,” issued Jul. 31, 2001. 6. U.S. Pat. No. 6,977,295 to Belotserkovskii, et al., issued Dec. 20, 2005, entitled “Locked nucleic acid hybrids and methods of use”.
U.S. Pat. No. 7,053,195 to Goff, entitled “Locked nucleic acid containing heteropolymers and related methods”, issued May 30, 2006.
U.S. Pat. No. 6,936,418 to Dutreix, et al., issued Aug. 30, 2005, entitled “Methods and compositions for effecting homologous recombination”.
WO/2001/083735, “Methods and compositions for effecting homologous recombination,” is a related application to Dutries, et al.
WO/1999/055916, “Methods, Kits and Compositions for Detecting and Quantitating Target Sequences,” published Nov. 4, 1999, by Boston Probes, Inc.
U.S. Pat. No. 7,229,767 to Kmiec et al., issued Jun. 12, 2007, entitled “Genomics applications for modified oligonucleotides”.
Hall et al., “Overview: Generation of Gene Knockout Mice,” Current Protocols Cell. Biol., published online, DOI: 10.1002/0471143030.cb1912s44, 1 Sep. 2009.

### BRIEF SUMMARY OF THE INVENTION

The following brief summary is not intended to include all features and aspects of the present invention, nor does it imply that the invention must include all features and aspects discussed in this summary.

The present invention comprises, in certain aspects, a method for effecting insertion of a DNA construct into a cell using recombination that is facilitated by the use of locked nucleic acids. In another aspect, the present invention comprises a method for altering gene expression by binding locked nucleic acids to a CCCTC-binding factor (CTCF) binding site.

In the first aspect, the present invention comprises a method for inserting a DNA construct into a target DNA sequence in a cell, said target DNA sequence being, for example a coding region within a gene, said sequence being double stranded and having a coding strand and a template strand. The method provides a hereditable change in the cellular DNA by combining an insert from the vector into the target and comprises the steps of providing in said cell: locked nucleic acid molecules having sequences complementary to at least a portion of said target genomic sequence on opposite template and coding strands, to form displaced strands; a vector comprising the DNA construct to be inserted and further comprising sequences complementary to said locked nucleic acids, whereby said locked nucleic acids bind to both the vector and the target genomic sequence; and a recombinase construct (i.e. the protein or a DNA expressing the recombinase) which combines said vector and the target genomic sequence, thereby causing insertion of the DNA construct into the target genomic sequence in the cell.

The DNA construct that is inserted into the cell may comprise a selectable marker to aid in isolation of cells that have taken up the construct. In one embodiment, the selectable marker is an antibiotic resistance gene. The DNA construct may further comprise a promoter for expressing the selectable marker. In another embodiment, the selectable marker may be detectable, e.g., a fluorescent protein.

The cell may be eukaryotic and may further be selected from the group consisting of mammalian and human.

In one embodiment, the method further comprises the step of providing in said cell locked nucleic acid molecules which comprise a pair of linked locked nucleic acids, i.e. linked together as in “Zorro” LNA.

In another embodiment, the step of providing in the cell a recombinase comprises introducing into said cell an expression vector expressing a specific recombinase. An example of a preferred recombinase is E. coli Rec A.

The locked nucleic acid molecules used in the present invention have sequences complementary to at least a portion of said target genomic sequence, said sequences being at least about 18 nucleotides in length. They will be complementary to a region on a plus (sense, or coding) strand and a region on a minus (anti-sense) strand.

The DNA construct to be inserted in the cell may block or alter gene transcription in the cell. The target genomic sequence may be a so-called “CCCTC-binding factor” (CTCF) binding site.

In one embodiment, the locked nucleic acid molecules and vectors are applied to more than one gene simultaneously.

In another aspect, the present invention comprises a method for suppressing gene expression of a selected gene in a genome of a cell, comprising the step of binding a pair of linked locked nucleic acids to a specific sequence that is a CCCTC-binding factor (CTCF) binding site that affects chromatin organization in the genome.

The gene may be of human origin. An example of a gene is a neurofibromatosis gene. The method may further comprise the step of identifying the binding site using chromatin immunoprecipitation.

The method may further comprise the step of identifying a CTCF binding site using a computerized CTCF binding site prediction tool and selecting a site within the locus of the gene and that has a unique sequence within the genome.

In a third aspect, the invention comprises a kit for modifying expression of a selected gene in a genome of a cell by altering a target region of a genomic sequence of the selected gene, comprising: (a) locked nucleic acid molecules having sequences complementary to at least a portion of the target region of the genomic sequence on opposite template and coding strands, to form displaced strands; and (b) a vector comprising the DNA construct to be inserted and further comprising sequences complementary to the locked nucleic acids, whereby the locked nucleic acids bind to both the vector and the target region.

The kit may further comprise a vector expressing a recombinase which combines the vector and the genomic sequence, thereby causing insertion of the DNA construct at the target region of genomic sequence in the cell.

The kit may further comprise vectors and locked nucleic acid molecules for a plurality of different genes.

### BRIEF DESCRIPTION OF THE DRAWINGS

FIG. 1A is a schematic representation of a DNA recombination mechanism using the present linked locked nucleic acid-mediated gene regulation (“LGR”) technology, in which portions of a vector are bound by the locked nucleic acid containing molecules and inserted into the target sequence. Two linked locked nucleic acid molecules are shown. In a preferred embodiment, these molecules are identical.

FIG. 1B is a schematic representation of a plasmid vector containing the two target regions, i.e. LNA-invaded g-DNA and LNA-invaded vector.

FIG. 2 is a diagrammatic representation of chromatin architecture and gene regulation altered by invasion of a linked locked nucleic acid (1-LNA). It shows a model of chromatin architecture at the NF1 locus. In normal fibroblast cell line GM01859, the ECR15 region remains close to ECR11 to form an ECR11-ECR15 loop. ECR15 is distant from ECR4, which facilitates NF1 expression. In linked LNA-treated cells, ECR15 is brought close to ECR4, which suppresses NF1 expression.

FIG. 3A is a schematic representation of chromosome 17, the NF1 transcripts, and the evolutionary conserved regions. FIG. 3B shows sequences of linked LNAs targeting an NF1 region as shown in FIG. 3A.

FIG. 4 is a representation of gels that show RT-PCR of NF1 expression in stable cell clones created by locked nucleic acid-mediated gene regulation (LGR) used to knock out NF1 expression. Clone N-1, F 2, 3, 4 and N were selected from pEEP-NF1E32N transfected GM00498; Clone NP and NP-1 were selected from pEEP-NF1E32NP transfected GM00498. Clone N was mixed colonies after picking up single colony. Clone NP was also mixed colonies after picking up single colony.

FIGS. 5A and 5B are bar graphs that show Q-PCR of NF1 expression in stable cell clones. A. Q-PCR result showed almost 100% suppression of NF1 using 3′-end primers #153/154. B. Q-PCR result showed less than 50% expression using 5′-end primers #157/158.

FIG. 6 is a diagrammatic representation of DNA structure of pEEP-NF1E32N (top) and pEEP-NF1E32NP (bottom), gene constructs used for insertion into the NF1 cell lines.

FIG. 7A show gels that show RT-PCR of CTCF expression in pEEP-CTCFE2 integrated 293T and GM00498 stable cells created using locked nucleic acid-mediated gene regulation. FIG. 7B shows integration of pEEP-CTCFe2 in the exon2 region of CTCF suppressed CTCF transcription completely.

FIG. 8A shows a gel that shows RT-PCR of mouse CTCF in pEEP-mCTCF integrated stable MBM cells. FIG. 8B shows a gel that illustrates RT-PCR of RB1 in pEEP-RB1E7 integrated stable GM00498 cells.

FIG. 9 shows a gel that shows RT-PCR of TP53 in pEGFP-TP53E6 integrated GM00498 stable cells.

FIG. 10 shows a series of gels that show RT-PCR of TP53 and NF1 in TP53-INF1-stable clones of GM00498 cells.

FIG. 11 is a schematic representation of ECRs (evolutionary conserved regions) at human NF1 locus. The schematic diagram of chromosome 17q11.2 shows the distribution of evolutionary conserved regions (ECR) around the NF1 locus. Distances from the transcription initiation site (TIS) are shown.

FIG. 12A is a gel showing specific amplification of ECR 15. FIG. 12B is a graph showing quantification of allele-specific expression of upstream 5′-end and downstream 3′-end transcripts of ECR15NF1 by real time-PCR.

FIG. 13 A is a photograph of a gel that shows altered protein-DNA interaction in linked LNA-treated GM01859 cells. ChIP assay using anti-CTCF MAb and anti-RNA Pol II MAb was used to detect CTCF binding and RNA Pol II binding in ECR regions after linked LNA treatment in GM01859 cells. +: linked LNA-treated cells, −: normal culture cell without linked LNA treatment. a ChIP assay showed reduced CTCF binding and RNA Pol II binding at ECR4, and increased CTCF binding and reduced RNA Pol II binding at ECR15 after linked LNA treatment. FIG. 13B is a photograph of a gel similar to FIG. 13A, namely a ChIP assay showing slightly increased RNA Pol II binding but no significant change in CTCF binding at ECR 11 after linked LNA treatment. FIG. 13C is a photograph of a gel that shows a CTCF binding site (B.S.) #3 region of human IGF2/H19 ICR served as control for CTCF binding in the ChIP assay.

FIG. 14A shows a gel of LNA-treated cells. FIG. 14B is a graph showing a decrease in activity in treated cells.

### DETAILED DESCRIPTION OF THE PREFERRED EMBODIMENT

### Definitions

Unless defined otherwise, all technical and scientific terms used herein have the same meaning as commonly understood by those of ordinary skill in the art to which this invention belongs. Although methods and materials similar or equivalent to those described herein can be used in the practice or testing of the present invention, the preferred methods and materials are described. Generally, nomenclatures utilized in connection with, and techniques of, cell and molecular biology and chemistry are those well known and commonly used in the art. Certain experimental techniques, not specifically defined, are generally performed according to conventional methods well known in the art and as described in various general and more specific references that are cited and discussed throughout the present specification. For purposes of clarity, the following terms are defined below.

The term “locked nucleic acid(s)”, or LNA, is used herein to refer to modified nucleotide(s) where the sugar ring is stabilized in a certain conformation. Typically, the ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. The locked ribose conformation enhances base stacking and backbone pre-organization. This significantly increases the hybridization properties (melting temperature) of oligonucleotides. LNA oligonucleotides are used to increase the sensitivity and specificity of expression in DNA microarrays, FISH probes, real-time PCR probes and other molecular biology techniques based on oligonucleotides.

An exemplary structure for an LNA nucleotide is shown below, from Torben Højland, et al. “LNA (Locked nucleic acid) and analogs as triplex forming nucleotides, Org. Biomol. Chem 2007, 5: 2375-2379, where T is thyminyl and R was varied in different analogs.

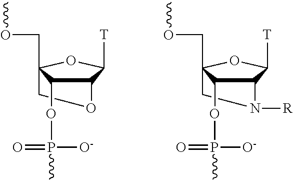

“Locked nucleic acid molecules” are oligonucleotides comprising locked nucleic acids; they comprise a 5′ end and a 3′ end, named with regard to the ribose structure, shown below:

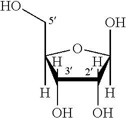

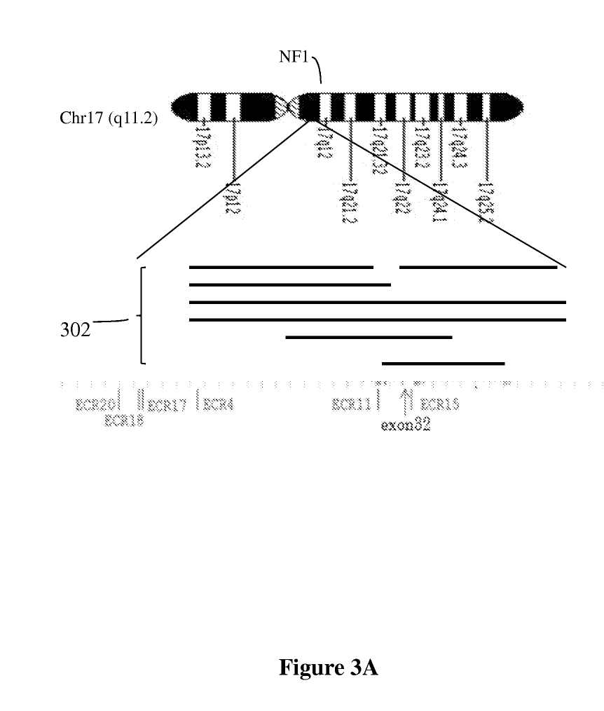

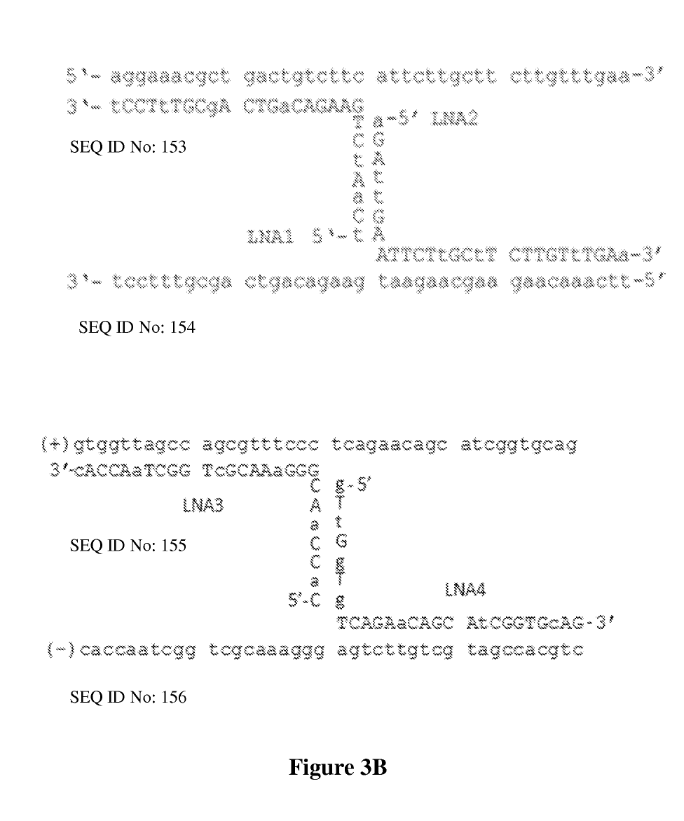

The present locked nucleic acid oligonucleotide molecules generally comprise a mixture of normal bases and locked bases. They may comprise range of percentages of locked bases. As can be seen in FIG. 3 and other sequences listed here, the present linked LNAs may in certain embodiments, contain, for example between 50% and 90% locked bases, or approximately 75% locked bases. The locked bases are shown in upper case in the sequences given. By way of example, the linked LNAs shown in FIG. 3 comprise about 20 bases in each single strand arm, and 7 bases in the “linker” portion, where hybridization occurs between portions of the two single LNA strands. 19/27 and 20/27 of the bases are locked nucleic acids. The LNA nucleotide ratios and sizes of the two single LNA strands and linker can be variable.

Alternative locked nucleic acid oligonucleotide molecules may be used, as long as they provide a degree of strand separation in their target sufficient to induce recombination with the vector. For example, triplex forming and quadruplex forming inserts are disclosed in U.S. Pat. No. 6,977,295 to Belotserkovskii et al., entitled “Locked nucleic acid hybrids and methods of use.” Other suitable LNAs are disclosed in U.S. Pat. No. 7,572,582 and U.S. Pat. No. 7,034,133, both to Wengel et al., entitled “Oligonucleotide analogues.”

The LNAs for use in the present invention may by synthesized or obtained from a variety of sources. For example, they may be made using standard phosphoramidite protocols on an ÄKTA Oligopilot (GE Healthcare). The DNA monomers may be obtained from Proligo (Sigma-Aldrich) and the LNA monomers and solid support may be obtained from Santaris Pharma (commercially available from Exiqon, Denmark).

The term “linked LNA” is used herein to refer to sequence-specific LNA oligonucleotides which simultaneously bind to both strands of a double stranded target, and also bind to each other. They can induce effective and specific strand invasion into DNA duplexes and potent alteration of gene expression, in a cellular context. The linked LNA comprise a major portion of locked nucleic acids and further comprise a linkage between two single stranded LNA oligonucleotides such that two free ends of the same polarity may be provided, i.e. two 3′ ends or two 5′ ends. An example of a linked LNA is a so-called Zorro LNA, where two strands are partially hybridized, as shown in FIG. 3B. Each strand has a portion binding to an exogenous DNA and a portion binding to a partner LNA. Another example is an arrangement of LNA molecules in a branched or cruciform shape. As described by Mui et al., “Convergent and General One-Step DNA-Catalyzed Synthesis of Multiply Branched DNA,” Org Lett. 2008 Oct. 16; 10(20): 4417-4420, a 15HA9 deoxyribozyme can be used to mediate nucleophilic attack of the 2′-hydroxyl group of a ribonucleotide embedded within one DNA substrate into a 5-adenylate of the second DNA substrate. This approach can be used to synthesize multiply branched DNA with a wide range of DNA sequences.

Also, branched chains of oligonucleotides can be created as described in US 2006/0286583, entitled “Multiplex branched-chain DNA assays.” Oligonucleotide dendrimers are described in Shchepinov et al., “Oligonucleotide dendrimers: stable nano-structures,” Nucleic Acids Research, 1999, Vol. 27, No. 15.

The present vector, which is used to provide sequences complementary to the 1-LNA and to provide an insertion sequence, may be a plasmid, virus, or any other genetic construct capable of being engineered with desired sequences, entering a cell and interacting with the genome thereof. Preferably, the vector is an expression vector to enable production of markers such as antibiotic resistance markers, fluorescence or optical markers or the like. The vector will be complementary to a target sequence, that is, will have sufficient sequence complimentarily to permit Watson-Crick base pairing between the vector and the I-LNAs. Similarly, the 1-LNAs will also be complementary to a genome sequence to be altered by insertion of the vector portion between the target regions.

The cells preferred for use in the present invention are eukaryotic, especially including human and animal cells where specific cell lines, especially knock-out cell lines, are desired for culture and study, or possibly for re-implantation into a subject.

As described below, selectable markers are used to isolate the desired cells. In a preferred embodiment, they prevent growth of cells that have not taken up the vector. The vector contains a gene that confers resistance to an agent to which the cells are exposed. Examples of selectable markers include, but are not limited to the DHFR resistance gene, which confers resistance to methotrexate (Wigler, et al., 1980, Natl. Acad. Sci. USA 77:3567; O'Hare, et al., 1981, Proc. Natl. Acad. Sci. USA 78:1527); the gpt resistance gene, which confers resistance to mycophenolic acid (Mulligan & Berg, 1981, Proc. Natl. Acad. Sci. USA 78:2072); the neomycin phosphotransferase gene, which confers resistance to the aminoglycoside G-418 (Colberre-Garapin, et al., 1981, J. Mol. Biol. 150:1); and the hygromycin resistance gene (Santerre, et al., 1984, Gene 30:147).

In addition, one may use screening markers to allow visualization of cells that have taken up the vector, such as by visual inspection of a culture of cells being treated with the 1-LNA. For example, marker genes may provide catalysis reactions resulting in a visible outcome (for example the production of a blue color when beta galactosidase is expressed in the presence of the substrate molecule 5-bromo-4-chloro-3-indoyl-β-D-galactoside) or confer the ability to synthesize particular amino acids (for example the HIS3 gene confers the ability to synthesize histidine). Other suitable screening markers include enhanced fluorescent proteins (e.g. GFP), beta galactosidase (to act on a chromogenic substrate) luciferase, etc.

The term “recombinase” means an enzyme or group of enzymes, or enzyme and auxiliary proteins that bring about sequence specific recombination between a donor sequence and an acceptor sequence, with partial homology between them. The terms “sequence-specific recombinase” and “site-specific recombinase” refer to enzymes or recombinases that recognize and bind to a short nucleic acid site or “sequence-specific recombinase target site”, i.e., a recombinase recognition site, and catalyze the recombination of nucleic acid in relation to these sites. These enzymes include recombinases, transposases and integrases.

A variety of recombinases may be used, provided they are specific for homology regions and result in inclusion of a homology donating strand from the vector as described here. Rec A is exemplified as the preferred recombinase. RecA has a structural and functional homolog in many species. The homologous protein in Homo sapiens is called RAD51. The Rec A used here may have sequence variation from a given native sequence. Characteristic of the L1 and L2 domains of E. coli Rec A (residues 157-164 and 195-209) is their high degree of conservation in prokaryotic cells. In a sequence of 64 bacteria the RecAs has 11 of the residues from 193 to 212 almost identical, and 6 are highly conserved, thus 17 over 20 residues are either identical or chemically conserved. On the other end, the eukaryotic homologues Rad51 and Dmc1 are considered to have an analogue function to the RecA. They share a strong homology sequence, in particular within the 230 amino acids in the core of the structure. Even though the loop L2 is in the core of the enzyme, and the residues that attach the loop to the whole enzyme are identical, some of the critical residues in L2 of RecA like Arg196 and Lys198 are absent in the corresponding region of the Rad51 and Dmc1. The recombinase used should carry out strand exchange. As described in West et al., “Mechanism of E. coli RecA protein directed strand exchanges in post-replication repair of DNA,” Nature 294, 659-662 (17 Dec. 1981), RecA protein promotes a reciprocal exchange of strands between paired DNA molecules. The mechanism of sister strand exchange thought to occur during post-replication repair was investigated, and it was shown that RecA protein initiates strand exchange from a nicked duplex, transferring the 3′-OH terminus at the nick into the single-stranded (ss) region of the gapped molecule. In the presence of ATP, two heteroduplex molecules are formed as RecA protein drives the reciprocal exchanges in one direction starting at the site of the original crossover.

### Overview

The present disclosure relates to the use of locked nucleic acids to alter gene expression by binding to a CTCF (CCCTC binding factor) binding site. It also relates to the use of locked nucleic acids to facilitate homologous recombination in a methodology termed here “LGR.” LGR employs linked LNAs and a recombinase.

### LGR Method (LNA Mediated Gene Regulation)

As disclosed herein, locked nucleic acid-mediated gene regulation (LGR) technology is a DNA recombination technology which employs (1) a pair of locked LNA oligonucleotides which target to a specific site in a genome, (2) a DNA construct containing the target region to be inserted and a homology region, and (3) a vector expressing a specific recombinase protein. Upon co-transfection of the locked LNA oligonucleotides and the other two constructs into a cell line, LNA oligonucleotides may induce DNA single strand formation at the target region of a specific gene at both alleles and in the construct with the target DNA region, the specific protein can bind to the regions and cut the single strand DNA region at the end, which may induce DNA recombination using DNA repair machinery in the cell to integrate the construct into the specific site of the genome. The present locked nucleic acid-mediated gene regulation (LGR) technology provides higher integration efficiency in different cell lines, and great site-specific integration in a shorter time period. LGR technology can be used to make kits for gene suppression, which may be used in any lab investigating gene function, and make stable cell lines for protein production, which can be used to develop new gene therapy strategies. LGR technology can replace RNAi technology for gene knockdown and ZFN technology for gene integration with much higher efficiency and specificity, which may facilitate basic research of functional genome studies and drug development. The kits can contain 1-LNAs that target a plurality of genes. For example, they may be targeted to an exon within the gene, and may be provided to target a large number of human genes, enabling the user to prepare knock-out cells for all genes in a set. It is possible in this way to prepare a kit containing 1-LNAs targeting all known human genes, as listed in the NCBI gene database, http (colon) (slash slash) www(dot)ncbi.nlm.nih.gov/gene/.

The present locked nucleic acid-mediated gene regulation (LGR) technology may be compared with currently used technology for gene silencing by RNAi technology or DNA insertion using ZFN technology (Table 1). LGR technology shows advantages in integration efficiency and gene suppression efficiency. For ease of selection of altered cell lines, LGR technology utilizes puromycin antibiotic resistance genes (or other selectable markers) and EGFP fluorescence (or other screening markers) to make positive clones that are easy to screen, and facilitate to obtain stable cell lines. This technology may provide researchers with a powerful tool for the study of gene function that can help elucidate mechanisms of disease development. Combined with other technologies, such as stem cell technologies, LGR technology can also be used to develop animal models for the study of human diseases, and to develop novel strategies for cancer therapy.

TABLE 1
Comparison of LGR technology and conventional technologies

| | Conventional | ZNF-driving | LGR |
|---|---|---|---|
| Attribute | gene targeting | targeting | targeting |
| Permanent disruption | Yes | Yes | Yes |
| Frequency in mammalian | 0.0001% | 1-20% | 0.1-30% |
| cell lines | | | |
| Time to biallelic knockout | 16 weeks | 4 weeks | <4 weeks |
| Screening | easy | difficult | easy |

The present LGR method utilizes cellular mechanisms that repair DNA double stranded breaks (DSB). By way of introduction, it is pointed out that the two major pathways are homologous recombination and DNA end-joining. The fundamental difference between these pathways is their dependence on DNA homology and accuracy of repair. In general, homologous recombination ensures accurate repair by using the un-damaged sister chromatid or homologous chromosome as a template. DNA end-joining, on the other hand, uses no or extremely limited sequence homology to rejoin ends in a manner that need not be error free. Both major pathways can be divided into subpathways that can result in different outcomes of DSB repair and might require common as well as subpathway-specific genes. DNA end-joining includes precise end-joining and micro-homology-directed end-joining, as described further in Sharma et al., “Nonhomologous DNA End Joining in Cell-Free Extracts,” J Nucleic Acids. 2010; 2010: 389129.

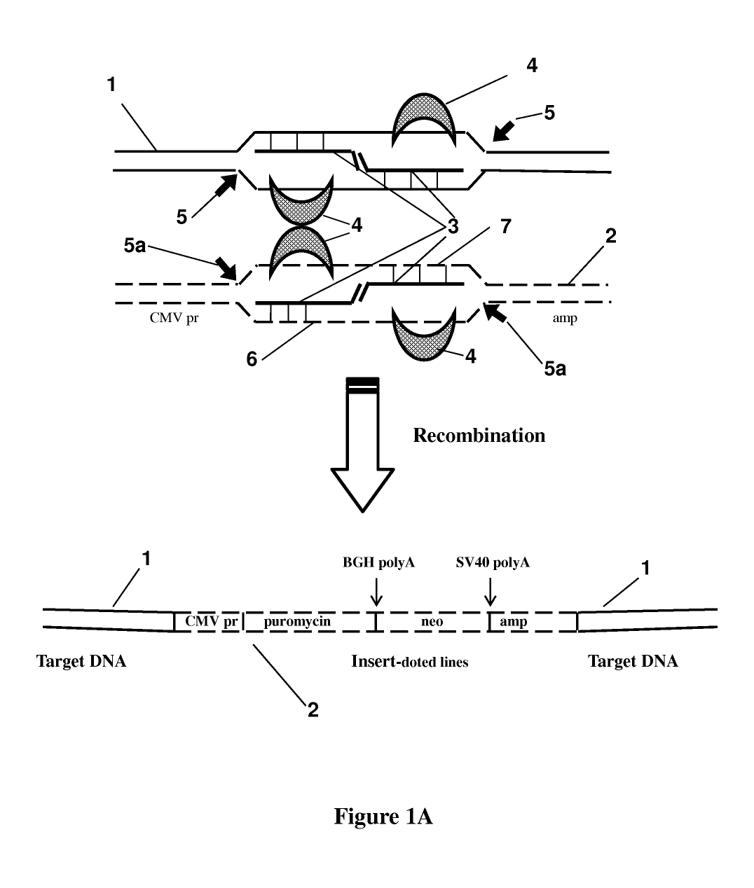

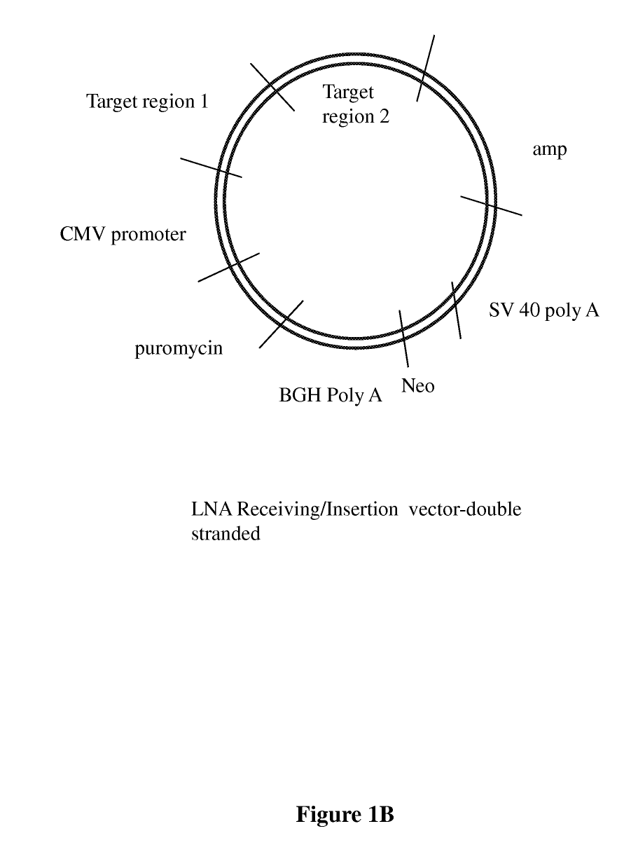

FIG. 1 illustrates a DNA recombination mechanism using this technology. A pair of LNA oligonucleotides 3 (e.g. in the form of a single linked locked LNA, or Zorro LNA) is introduced into a cell and combine with two different DNA molecules. One molecule is DNA already present in the cell, such as genomic DNA in a chromosome, which is present in double stranded form, and may be present in multiple copies. The other molecule which is also complementary to and binds to the LNA oligonucleotides is a double stranded DNA in a vector, shown by broken lines. The vector may contain disrupting sequences, such as BGH poly A and selectable markers, such as neomycin, ampicillin, and puromycin. These coding sequences are immediately adjacent to the sequences where the LNAs bind, within a distance that crossing over from the vector to the target can occur. Also introduced is a vector expressing a recombinase protein 4 and a vector 2 containing the target region that combines with LNA. A recombinase 4 dimer is shown binding to both the vector 2 and target DNA 1. The recombinase will separate the dsDNA into single strands in a portion of the DNA where the linked locked LNA hybridizes. The vector 2 will also be separated into single strands in a portion, since, as stated, it contains a portion that hybridizes to the same LNA oligonucleotides 3 that hybridize to the target DNA 1, indicated as target regions 6, 7. The recombinase 4 acts on both the target DNA 1 and the vector 2. The pair of LNAs which forms the linked LNA 3 in both the target DNA 1 and the vector 2 induces single strand DNA formation at the target region in the genome 1 and single strand formation in the introduced vector 2. The LNAs 3 are shown as being inserted in to both the target DNA 1 and the vector 2. The recombinase protein 4 binds to the target regions and introduces nicks in the DNA at specific points 5 flanking the LNAs. Single strand binding also will likely involve other cellular proteins, such as single stranded binding protein. This binding of proteins to the vector 2 and the target DNA 1 leads the DNA repair machinery in the cell to induce DNA recombination. As a result, the vector construct is integrated at the specific site in the genome. As shown in the bottom portion of FIG. 1, the exemplary vector 2 contained an insert, comprising a CMV promoter, a puromycin resistance gene, a BGH poly A signal, a neomycin marker, and SV40 polyadenylation signal, and an ampicillin resistance gene. This insert was contained between the hybridizing portions of the vector 2; that is the vector 2, which preferably is in the form of a plasmid, would have an orientation in which the insertion sequence is flanked by the two target regions, as shown in FIG. 1.

The LNAs comprising the linked LNA may be of a variety of lengths, e.g. between 15 and 30 nucleotides in length, or between 15 and 40 nucleotides in length. An LNA oligonucleotide may contain variable number of locked nucleotides. The LNAs comprising the linked LNA are complementary to the sense and antisense strands of DNA at the target region. Approximately 5-10 nucleotides, including locked nucleotides of the 3′ end of one LNA, are complementary to 5-10 nucleotides of the 5′ end of the other LNA, thus forming the linker portion of “linked LNA”. The linked LNAs are introduced into a cell by any of the known methods of introducing nucleic acids in cells, including microporation and transfection. The-linked LNAs are introduced into a cell along with a vector containing the target region and a vector encoding a recombinase protein. The linked LNA invades the target region contained in the vector as well as in the genome and causes DNA conformation change. The recombinase protein binds to the region with conformational change and induces nicks in the DNA. Such breakage formed at the locus induces the cell's DNA repair mechanism. At the same time, the recombinase protein anchors the vector containing the target region near the genome and mediates recombination such that the target region contained in the vector is integrated into the corresponding region in the genome. The integration of the vector containing the target region leads to enhanced or suppressed expression of the target gene.

The LGR technology is used in the efficient production of stable cell lines with altered gene expression. Gene expression can be tuned by targeting the exon region or by targeting the promoter region of a gene to decrease gene expression. Alternatively a silencer region of a gene can be targeted to enhance gene expression. Long Range Gene Modulation may be used here. The technology is useful for regulation of lethal or housekeeping genes. The technology is also efficiently used to introduce two or more gene mutations to study gene interaction and cell signaling in complex disease development. The technique targets a single site in the genome, achieving a better sequence specificity than the prevalent technologies such as zinc finger nuclease technology or RNA-targeted techniques. The technology disclosed herein also is more efficient and gives faster results than the above techniques (See Table 1 above).

### Long Range Gene Modulation by Affecting DNA Binding Proteins

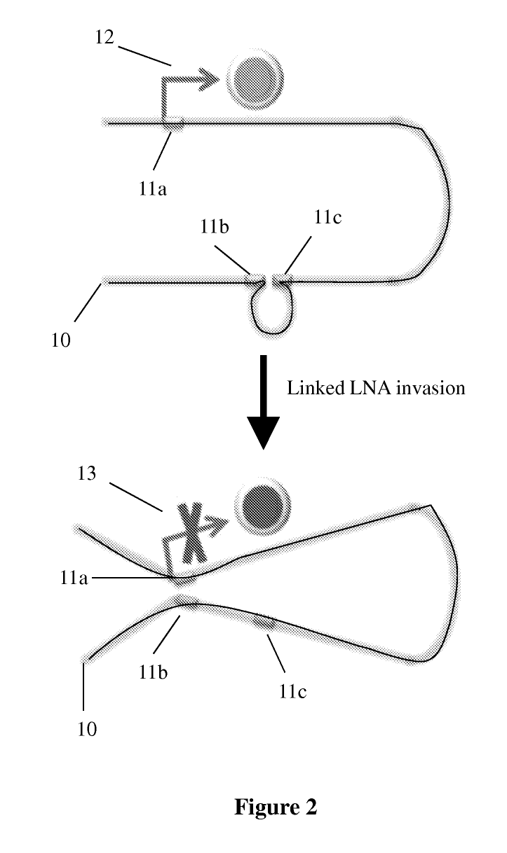

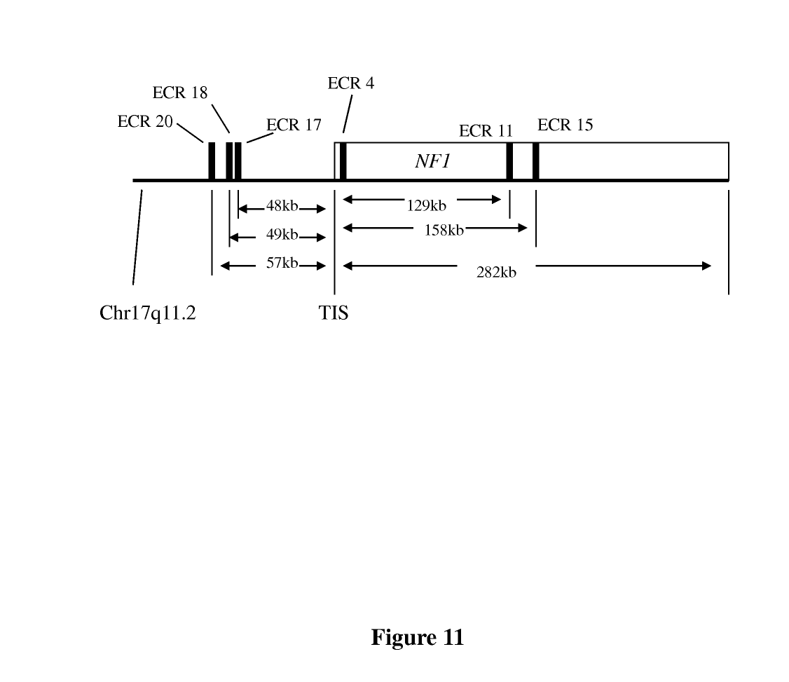

Another embodiment of the invention is described in FIG. 2. FIG. 2 shows the mechanism by which linked LNA alters gene expression via long range DNA interactions. A gene contained in a genome 10 has one or more cis elements which may enhance or suppress gene expression (11a, 11b, 11c) arranged at various sites relative to the structural gene (as shown in FIG. 11). A cis element may be an intergenic enhancer, an endodermal enhancer, a mesodermal enhancer, a coactivator, an imprinting control region or a gene silencer. In one embodiment, the cis element is a region which binds a zinc finger DNA binding protein. In FIG. 2, cis element 11a is situated near the promoter of the gene. In the absence of linked LNA invasion, elements 11b and 11c are tied in a long range DNA interaction and transcription of the gene ensues, shown at 12. Upon introduction of linked LNA targeted to cis element 11a, long range DNA interaction is mediated by which silencer element 11b is juxtaposed close to the promoter region of the gene and gene expression is suppressed, as shown at 13.

Long range intra- and inter-chromosomal interactions establish a nuclear architecture that participates in the regulation of gene transcription. CCCTC-binding factor (CTCF), a ubiquitously expressed and evolutionarily conserved 11-zinc-finger DNA binding protein, has been implicated as the glue that maintains these interactions in the three-dimensional nuclear space. More than 26,000 CTCF binding sites in the human genome were recently identified using ChIP-chip and ChIP-seq technologies. CTCF has been implicated in numerous regulatory functions, including insulation, genomic imprinting, X-chromosome inactivation, transcriptional activation and repression and mediation of long-range DNA interactions. To achieve these seemingly unrelated functions, CTCF can interact with a variety of proteins, including RNA Pol II and Kaiso methyl-CpG-binding protein. Moreover, CTCF itself is modified by phosphorylation, poly (ADP-ribosyl)ation and SUMOyation to regulate gene expression. The central role of CTCF in establishing higher-order chromatin organization through the establishment of long-range DNA interactions involves diverse partners, including cohesins, Yy1, and polycomb repressive complex 2 (PRC2). These CTCF-containing protein complexes regulate the interplay between DNA methylation, higher-order chromatin structure and lineage-specific gene expression.

The ability of CTCF to modulate chromatin tertiary conformation and to regulate gene expression has been extensively examined at the Igf2/H19, β-globin, and human major histocompatibility complex class II (MHC-II) loci. At the mouse imprinted Igf2/H19 region, the maternally expressed H19 gene is located approximately 80 kb downstream from the paternally expressed Igf2 gene. The imprinting control region (ICR), located 2-4 kb upstream of H19, contains 4 CTCF-binding sites which may interact with other cis-elements, such as the differentially methylated region-1 (DMR1) in the promoter region of Igf2, the DMR2 within Igf2 exon 6, an intergenic enhancer, an endodermal enhancer and a mesodermal enhancer downstream of H19. Methylation of these binding sites blocks CTCF binding on the active paternal Igf2 allele, giving the various enhancers direct access to the Igf2 promoters. On the other hand, binding of CTCF to the maternal unmethylated ICR results in the maternal Igf2 allele forming a complex three-dimensional knotted loop that keeps the enhancers away from the Igf2 promoters, thereby silencing the maternal Igf2. Mutation or deletion of the ICR, CpG methylation at CTCF binding sites in the ICR that abrogate CTCF binding, and knockdown of CTCF by RNAi may change these reciprocal interactions, affect DNA tertiary conformation and ultimately alter Igf2/H19 imprinting.

The present examples of Long Range Gene Modulation involve the NF1 gene. Neurofibromatosis 1, one of the most common autosomal dominant disorders, is caused by deletions or mutations in the gene NF1 on human chromosome 17q11.2. NF1 extends over a ˜282 kb DNA region and encodes neurofibromin, a cytoplasmic protein that is initially ubiquitously expressed, suggesting that the gene subserves a housekeeping function. Neurofibromin inhibits cell growth and acts as a tumor suppressor by inactivating RAS proteins, which are proto-oncogene products. We have shown that there is a CTCF-dependent long-range association between Nf1 on mouse chromosome 11 and Igf2 on mouse chromosome 7, and that the transcription of both genes is altered when CTCF is knocked down. In this study, we were interested in examining the role of CTCF in the regulation of human NF1 long-range interactions and gene expression.

The linked LNA used in this study employed two 20-mer arms to locate its genomic DNA target site. In theory, it targets a single site in the human genome, achieving a better sequence specificity than RNA silencing methods. linked LNA methodology provides an efficient, convenient and rapid method to manipulate gene expression, although we cannot rule out the possibility of an off-target effect using linked LNA to regulate gene in a large genome.

The examples below show how the linked LNA was adapted technique to study NF1 gene regulation by targeting a single CTCF binding site in a human fibroblast cell line GM01859. The data indicate that linked LNA binding to the target CTCF binding site facilitated CTCF binding, altered long-range chromatin interactions at the NF1 locus, and down-regulated NF1 expression. This study illustrates an effective and convenient technique to alter DNA long-range interactions and thereby manipulate gene expression without altering total DNA content or linear sequence.

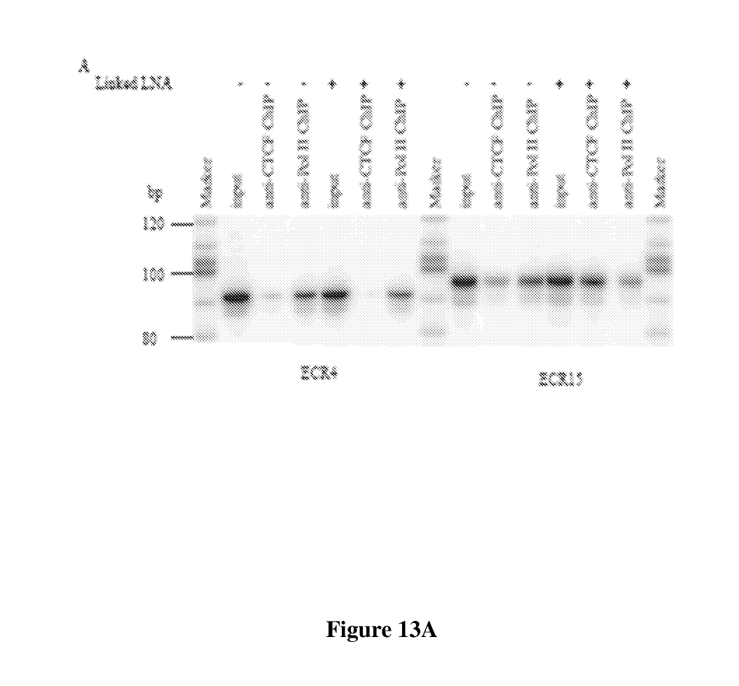

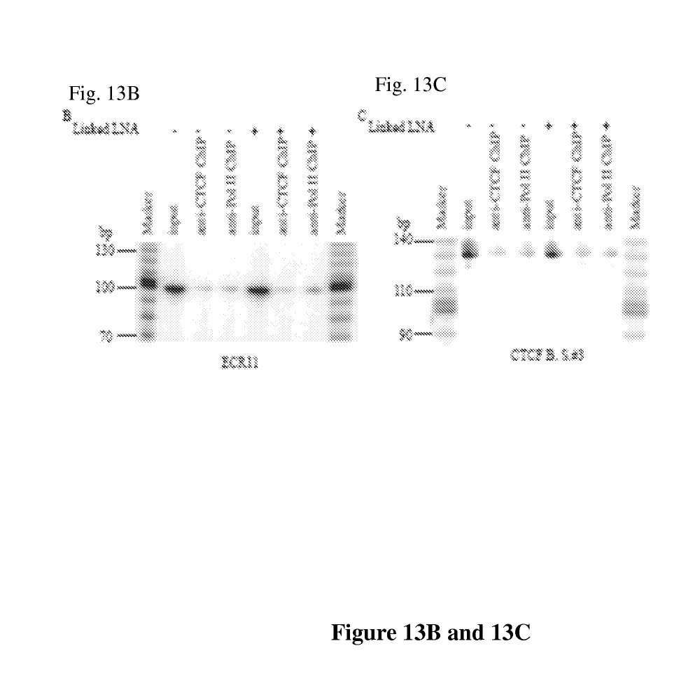

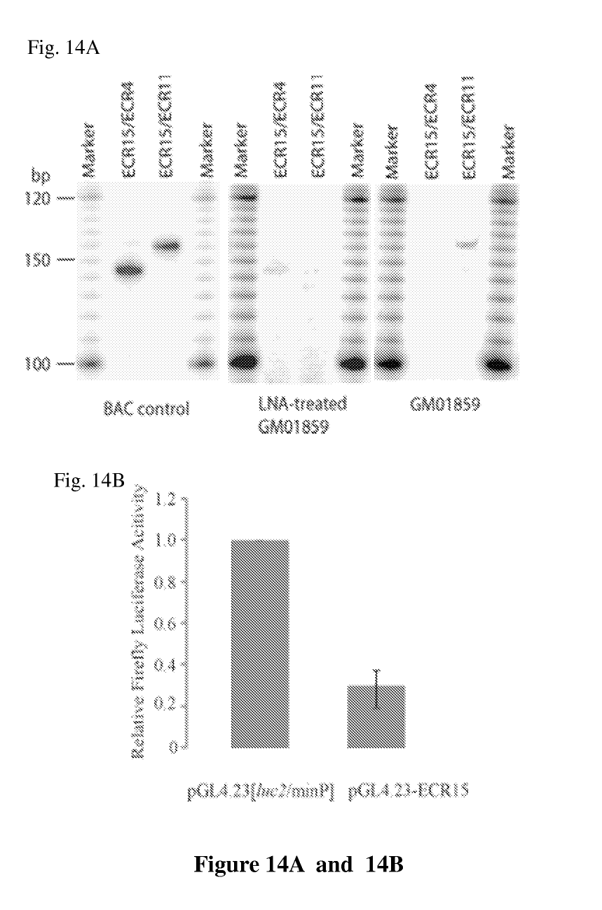

As shown below, in cell culture, linked LNA binding altered the long-range DNA interaction between ECR15 and ECR4 and ECR11 (FIG. 14; FIG. 2). This alteration was accompanied by a change in protein-DNA interactions. Altered binding of CTCF and RNA Pol II was observed specifically at regions ECR15, ECR4 and ECR11 (FIG. 13A-B), but not at ECR17, ECR18, ECR20 and CTCF binding site 3 of H19. Linked LNA targeted to the ECR15 CTCF binding site down-regulated NF1 expression and altered long-range DNA interactions with ECR4 and ECR11, but it did not affect the unrelated remote DNA regions, ECR17, ECR18 and ECR20. L-LNA targeted to ECR15 increased CTCF binding at ECR15, and increased RNA Pol II binding at ECR11, but decreased CTCF binding at the ECR4 region. These changes could not be explained by the local DNA configuration of linked LNA-targeted ECR15, as it affected protein-DNA interactions at distant regions, such as, ECR4 and ECR11. Gene expression from both of the two NF1 alleles decreased in the linked LNA-treated cells, demonstrating that linked LNA could target the two alleles with similar efficiency. linked LNA binding to ECR15 may result in a change in CTCF binding with ECR4 and ECR15, leading to further changes in chromatin architecture which drove the silencer ECR15 closer to the promoter region of NF1 (FIG. 14B-FIG. 2) and suppressed its expression.

In FIGS. 14A and B is shown a graph and diagram that show long-range DNA interactions between ECR15 and ECR4 or ECR11 in GM01859 cells. (top). Long-range DNA interactions between ECR15 and ECR4 were detected in linked LNA-treated GM01859 cells but no interaction was detected between ECR15 and ECR11. In normal fibroblasts, an interaction between ECR15 and ECR11 was detected, but no interaction was seen between ECR15 and ECR4. Random re-ligated BAC clone RP11-1107G21 DNA after Hind III digestion served as control. (bottom). Functional analysis of ECR15 region with Dual-Luciferase Reporter Assay System revealed ECR15 region may suppress gene expression of firefly luciferase in pGL4.23-ECR15-transfected Hela cells compared to the control cells, which were transfected with pGL4.23[luc2/minP]. Values are means±SD from the four repeats.

Since CTCF acts as the glue that maintains intra- and inter-chromosomal interactions, disruption of CTCF binding at a targeted DNA site may result in changes in DNA long-range interactions, leading to activation or repression of gene expression at a specific locus. A mutation of a CTCF binding site at the ICR of the mouse Igf2/H19 imprinting locus abrogated CTCF binding, changed the local chromatin architecture, and induced loss of Igf2 and H19 imprinting. Tanimoto et al showed that the inversion of LCR (HS1 to HS5) in a human β-globin yeast artificial chromosome transgenic mouse significantly diminished expression of all β-globin genes in a CTCF- and developmental stage-dependent manner. Splinter et al showed that both conditional deletion of CTCF and a targeted disruption of a DNA-binding site destabilize the long-range interactions at the locus. Gene modulation, either by homologous recombinant or ZFN technology to insert, delete or site-mutate a target DNA, actually changes the DNA content, and may alter genome integrity. Linked LNA does not affect the target DNA content or genome integrity.

Thus there is provided a novel method to manipulate gene expression through alteration of long-range DNA interactions, leaving the DNA sequence intact. The present methods make possible diagnosis and treatment of microdeletion syndromes involving long range interactions and loss of chromatin architecture. In some microdeletion syndromes, long range DNA interactions may be disrupted, leading to changes in the transcription of genes distant from the gene suffering the deletion. Approximately 5 to 10% of all NF1 patients carry a heterozygous deletion of approximately 1.5 Mb involving the NF1 gene and contiguous genes lying in its flanking regions. This NF1 microdeletion syndrome is often characterized by a more severe phenotype than that observed in the majority of NF1 patients. In particular, patients with NF1 microdeletion often show variable facial dysmorphism, mental retardation, developmental delay, and an excessive number of neurofibromas for age.

### EXAMPLES

### Examples 1-5 Pertain to L-LNA Mediated Gene Regulation; Examples 6-8 Pertain to Long Range Gene Modulation

### Example 1

### Materials and Methods

Cell Culture.

Fibroblast cell line GM00498 was purchased from CORIELL Institute for Medical Research, and was cultured in minimum essential medium (MEM) supplemented with 1× non-essential amino acids (NEAA) and 15% fetal bovine serum (Invitrogen). Five μg/ml of Plasmosin (InvivoGen) was also added to the culture medium to protect the cells from mycoplasma contamination. 293T cells (ATCC) and a mouse primary fibroblast cell MBM, derived from mouse newborn skin tissue in our laboratory, were cultured in Dulbecco's modified Eagle's medium (DMEM) (Invitrogen) supplemented with 10% fetal bovine serum and 5 μg/ml of Plasmocin. All cell lines were grown at 37° C. with 5% of CO₂.

Linked LNA Preparation.

LNA oligonucleotides were synthesized by the PAN facility, Stanford University, and dissolved in sterile Milli Q water at 100 mM stock concentration. Linked LNA is prepared as described below. Briefly, for each transfection, five μl of 100 μM LNA 1 and 5 μl of 100 μM LNA2 were mixed in a 200-μl PCR tube with 1 μl of 150 mM sterile PB, pH7.4, covered by 15 μl of Chill-Out liquid wax (Bio-Rad, CA). The solution was denatured at 90° C. for 2 min in an Eppendorf thermo cycler (Mastercycler gradient). The cycler was then turned off to let the LNA solution cool down to 37° C. to form a linked LNA structure. The solution was centrifuged, the liquid wax was removed, and the linked LNA solution stored at 4° C. overnight, kept ready for transfection. 5 μl of linked LNA was mixed with 1 μl of 1 μg/μl gene-specific construct and 0.6 μl of 150 mM sterile PB, pH 7.4, and covered with 15 μl of liquid wax in a 200-μl PCR tube. The solution was incubated at 37° C. overnight, and the liquid wax was removed immediately prior to transfection.

Cell Transfection.

Cells for microporation were prepared following the protocol provided by the vendor (Invitrogen, CA). Trypsinized GM01859 cells were collected, counted and centrifuged at 700 rpm for 5 minutes at room temperature, and the cells were resuspended in 2 ml of 1×DPBS (Invitrogen, CA). 1˜1.5×10⁶ cells were transferred into two 1.5-ml microtubes and spun again for 5 minutes under the same conditions. The cells were resuspended in 100 μl of resuspension solution provided by Invitrogen after removing the 1×DPBS. Five μl of linked LNA, five μl of linked LNA-bound gene-specific construct (1 μg), and 1 μl of 1 μg/μl recombinase-expressing construct were mixed with the cells by gentle pipetting. Microporation was performed in a 100-μl tip at 1,400 volt for 20 milliseconds in a MicroPorator MP-100 (Invitrogen, CA). The transfected cells were incubated in three wells of one 6-well plate with 2 ml of medium described above without Plasmocin. After for 24-hour incubation, five μg/ml of Plasmocin were added into each well, 1 μg/ml of puromycin (Invivogen) was also added into each well for selection for 2 days; the puromycin concentration was changed to 0.4 μg/ml for maintenance. When using G418 for selection, the working concentration was 1-1.2 mg/ml for 1 week, then changed to 0.4 mg/ml for maintenance. After culturing and selection for two weeks, colonies formed and are ready for pickup; each colony may be cultured in one well of a six-well plate with medium described above and 0.2 μg/ml of puromycin or 0.2 mg/ml of G418 and 1 μg/ml of Plasmocin.

Quantification of Gene Expression by Real Time-PCR and RT-PCR.

Cells were lysed in 1 ml of Tri Reagent (Sigma-Aldrich) after removing the culture medium and washing by 1 ml of 1×DPBS. Total RNA was purified by following the protocol provided by the vendor. 1 ml of lysate was collected into 1.5-ml tube, 0.2 ml of chloroform was added and mixed thoroughly by shaking several times. After spinning for 15 min at 12,000 rpm for 15 min, the supernatant was collected and 0.65 ml of isopropanol was added to the supernatant, mixed thoroughly and spun at 12,000 rpm for 10 min. The supernatant was discarded, 0.8 ml of 75% ethanol was added, and the solution was centrifuged at 10,000 rpm for 10 min. The supernatant was discarded and the RNA pellet was allowed to air dry for up to than 3 min. The RNA was dissolved in 100 μl of sterile Milli Q water. RNA concentration was measured in NanoDrop 1000 Spectrophotometer (Thermo Scientific). One μg of total RNA was used to synthesize cDNA using SuperScript III First-Stand Synthesis SuperMix kit (Invitrogen) following the protocol provided by the vendor. Briefly, 1 μg of total RNA was mixed with 1 μl of 50 ng/μl of random hexamers, and 1 μl of annealing buffer. Sterile Milli Q water was added to 8 μl, and the contents were covered by adding 15 μl of liquid wax. The mixture was incubated in a thermal cycler at 65° C. for 5 min, and immediately placed on ice for 1 min. The contents of the tube were collected by brief centrifugation, and 10 μl of 2× first-strand reaction mix, and 2 μl of SuperScript III/RNAseOUT enzyme mix were added. The sample was mixed by pipetting and collected by brief centrifugation. The mixture was incubated at 25° C. for 10 min, followed by incubation at 50° C. for 50 min. The reaction was terminated by incubation at 85° C. for 5 min, followed by incubation on ice with the addition of 60 μl of sterile Milli Q water after the removal of the liquid wax. RT-PCR reaction was made by mixing 2 μl of cDNA, 1.5 μl of 3× KlenTaq DNA polymerase mix (containing 0.8 unit of KlenTaq DNA polymerase, 0.75 mM dNTP, 3× buffer), and 1 μl of 10 μM of primer pairs. The following thermo cycle for RT-PCR with KlenTaq DNA polymerase (Ab Peptides Inc.) was used: 95° C. 2 min, 36 cycles of 95° C. 20 sec, 63° C. 30 sec and 72° C. 20 sec, followed by 5 min extension at 72° C. PCR products were subjected to electrophoresis and analysis using MultiNA (Shimadzu, Japan). Primers are listed in Table 2.

Real time-PCR assays for gene expression were run in quadruplicate in a 384-well plate using an ABI Prism 7900HT, SYBR Green and the ABI protocol. Relative levels were determined by a “delta Ct and delta-delta Ct” with reference to a human β-actin control using primer pairs #5899 (SEQ ID No: 56) and #5900 (SEQ ID No: 57). All of the primers are listed in Table 2, which were synthesized in Pan Facility, Stanford University.

TABLE 2
List of oligonucleotides used

| SEQ | | | |
|---|---|---|---|
| ID | Oligo | | |
| No. | # | Sequence | Memo |
| 1 | 361 | TagcgtttaaacttaagcttATGGTGAGCAAGGGCGAG | For cloning of EGFP into |
| | | GAGCTG | pEXoIN at Hind III site |
| 2 | 362 | TcggtcatggtaccaagcttCTTGTACAGCTCGTCCAT | |
| | | GCCGAGAGT | |
| 3 | 367 | GTGGTTAGCCAGCGTTTCCCTCAGAACAGC | As PCR template of NF1 exon |
| | | ATCGGTGCAG | 32 region |
| 4 | 368 | tcaagtgtatcatatgGTGGTTAGCCAGCGTTTCCC | For cloning of NF1 exon 32 |
| 5 | 369 | gcgtacttggcatatgCTGCACCGATGCTGTTCTGA | region into Nde I site of pEEP |
| 6 | 382 | GaccggcgcgccggatccATGGCTATCGACGAAAAC | For cloning of RecA at BamH I |
| | | AAACAG | site of pEXoIN |
| 7 | 383 | CactggactagtggatccTTAAAAATCTTCGTTAGTT | |
| | | TCTGCTACG | |
| 8 | 370 | gcaggaaagaacatgtGTGGTTAGCCAGCGTTTCCC | For cloning of NF1 exon 32 |
| 9 | 371 | ccttttgctcacatgtCTGCACCGATGCTGTTCTGA | region at Pci I site of pEEP |
| 10 | 375 | GCAGAGCTCTCTGGCTAACTAG | For sequencing of insert at |
| | | | Hind III site of pEXoIN |
| 11 | 376 | GACGCACCCTGTCTGACTAC | For sequencing of insert at |
| | | | BamH I site of pEXoIN |
| 12 | 377 | AATGACGTATGTTCCCATAGTAACG | For sequencing of insert at Nde |
| | | | I site of pEEP |
| 13 | 378 | GCTTCCTCGCTCACTGACTC | For sequencing of insert at Pci I |
| | | | site of pEEP |
| 14 | 394 | Atacgtagatgtactgccaagtagg | Use with JL377 to screen |
| | | | positive clone inserted at Nde I |
| | | | site of pEEP by PCR |
| 15 | 395 | cctctgacttgagcgtcga | Use with JL378 to screen |
| | | | positive clone inserted at Pci I |
| | | | site of pEEP by PCR |
| 16 | 446 | TATTAAACTCTCACCTCCCATGTTGCTCAAA | As PCR template for cloning |
| | | GAACCATAT | RBI exon 7 region |
| 17 | 447 | tcaagtgtatcatatgTATTAAACTCTCACCTCCCA | For cloning of RB1 exon 7 |
| 18 | 448 | gcgtacttggcatatgATATGGTTCTTTGAGCAACA | region at Nde I site of pEEP |
| 19 | 451 | CAAGATGCGCTAGTGGACAGATTGCTGACC | As PCR template for cloning of |
| | | AGGGGCTTGA | CTCF exon 2 region |
| 20 | 452 | tcaagtgtatcatatgCAAGATGCGCTAGTGGACAG | For cloning of CTCF exon 2 |
| 21 | 453 | gcgtacttggcatatgTCAAGCCCCTGGTCAGCAATC | region at Nde I site of pEEP |
| 22 | 454 | AGCCTGTGGAGCGATTAAACCGTGCGCGGA | As PCR template for cloning of |
| | | GCTGCTTCTT | CTCF promoter region |
| 23 | 455 | tcaagtgtatcatatgAGCCTGTGGAGCGATTAAACC | For cloning of CTCF promoter |
| 24 | 456 | gcgtacttggcatatgAAGAAGCAGCTCCGCGCAC | region at Nde I site of pEEP |
| 25 | 465 | CACTTTTCGACATAGTGTGGTGGTGCCCTAT | As PCR template for cloning of |
| | | GAGCCGCCT | TP53 exon 6 region |
| 26 | 466 | TGCATTAGTTATTAATCACTTTTCGACATAG | For cloning of TP53 exon 6 |
| | | TGTGGTG | region into pEGFP N1 |
| 27 | 467 | ATTGATTACTATTAATAGGCGGCTCATAGG | |
| | | GCAC | |
| 28 | 468 | GCGTTACTATGGGAACATACGTC | For screening positive clone of |
| 29 | 469 | CTGCGTTATCCCCTGATTCTG | pEGFP-TP53 |
| 30 | 153 | CTAAAGAAGGTTGCGCAGTTAGCAG | Primers for Q-PCR and RT- |
| 31 | 154 | GCTGCTTTACGTTTGGTGCTTTCAG | PCR of NF1 |
| 32 | 157 | TAAGGACTCGCCTCTGCACAAAG | Primers for Q-PCR and RT- |
| 33 | 158 | ACTTCCTCTGGACTCTTGTCATTG | PCR of NF1 |
| 34 | 435 | cagcttgccgtaggtggcatc | Primers for 1^{st} round of inverse |
| 35 | 437 | GTCACCGCCGACGTCGAG | PCR |
| 36 | 440 | CCGGTGCCGGATCTATGCAGA | Primers for 2^{nd} round of inverse |
| 37 | 441 | gctgaacttgtggccgtttacgtc | PCR |
| 38 | 472 | GGAGGACCTGCCTCTCGTCAG | For RT-PCR of RB1. |
| 39 | 473 | ACCTCCCAATACTCCATCCACAGATG | |
| 40 | 478 | CGCTTCGAGATGTTCCGAGAGCTG | For RT-PCR of TP53 |
| 41 | 479 | GTCAGGCCCTTCTGTCTTGAACATG | |
| 42 | 3328 | aaggtgccacagacacagacttag | For RT-PCR of mouse CTCF |
| 43 | 3329 | tttcacatgggtgtccatgactg | |
| 44 | 3332 | tctaagtgtgggaaaacatttaca | For RT-PCR of human CTCF |
| 45 | 3333 | tccacgtttactcttcttcgtttct | |
| 46 | 274 | CaCCaACGGGaAACGcTGGCTaACCAc | LNAa of NF1 exon32 region |
| 47 | 275 | gTtGgTgTCAGAaCAGCAtCGGTGcAG | LNAb of NF1 exon32 region |
| 48 | 400 | tCaAtCtGTTtAAtCGCtcCACaGGCT | LNAa of CTCF promoter |
| | | | region |
| 49 | 401 | aGAttGaCGTgCgCGGaGCTGcTTCtT | LNAb of CTCF promoter |
| | | | region |
| 50 | 444 | CaCCaACTGgGAGgTGAgAGTtTAAtA | LNAa of RB1 exon7 region |
| 51 | 445 | gTtGgTgTGTtGCTCaAAGaACCaTAT | LNAb of RB1 exon7 region |
| 52 | 449 | CaCCaACCTgTCCaCTAgCGCaTCTtG | LNAa of CTCF exon2 region |
| 53 | 450 | gTtGgTgATTGcTGAcCAGgGGCtTGA | LNAb of CTCF exon2 region |
| 54 | 463 | CaCCaACCCaCACtATGtCGAaAAGaG | LNAa of TP53 exon6 region |
| 55 | 464 | gTtGgTgTGGtGCCcTATGaGCCgCCT | LNAb of TP53 exon6 region |
| 56 | 5899 | caggtcatcat/catc/tggcaatgagc | For RT-PCR of β-actin |
| 57 | 5900 | cggatgtca/cacgtcac acttcatga | |
| 58 | 1266 | CGAAAGGCAAGGAGGAAGCTTATCT | For RT-PCR of L7 |
| 59 | 1267 | CGAATTTCAG TTCTGTACATCTGCCT | |

### Plasmid Construction

a. pEEP Construction

pEGFP-N1 (BD Bioscience) was used as the template, and primer pair #361/362 was used to amplify EGFP ORF with Phusion DNA polymerase (NEB Biolabs). The EGFP ORF fragment was cloned into Hind III site of pEXoIN (gifted by Konstantin Matentzoglu, Martin Scheffner and Trenzyme Biotechnology GmbH) with Choo-Choo Cloning kit (MCLAB, CA) to form a fusion gene of EGFP and puromycin that may facilitate double screening. The new construct was named pEEP, and the insert was confirmed by sequencing using oligonucleotide #375. The purified plasmid DNA was prepared using HiSpeed Plasmid Maxi kit (QIAGEN).

b. pEEP-NF1E32N Construction

Using a primer pair #368/369 and oligonucleotide #367 as template, the fragment which corresponds to NF1 exon 32 region was amplified with KlenTaq DNA polymerase. The DNA fragment was inserted into Nde I site of pEEP with Choo-Choo Cloning kit. Primer pair #377/394 was used to screen positive clones by PCR using KlenTaq DNA polymerase. The construct was named pEEP-E32N, and the insert was confirmed by sequencing using oligonucleotide #377. The purified plasmid DNA was prepared using HiSpeed Plasmid Maxi kit (QIAGEN).

c. pEEP-NF1E32NP

Using a primer pair #370/371 and an oligonucleotide #367 as template, the fragment which corresponds to NF1 exon 32 region was amplified using KlenTaq DNA polymerase. The DNA fragment was inserted into Pci I site of pEEP-NF1E32N with Choo-Choo Cloning kit. Primer pair #378/395 was used to screen positive clones by PCR using KlenTaq DNA polymerase. The construct was named pEEP-NF1E32NP, and the insert was confirmed by sequencing using oligonucleotide #378. The purified plasmid DNA was prepared using HiSpeed Plasmid Maxi kit (QIAGEN).

d. pEXoIN-RecA

The RecA ORF region was amplified with Phusion DNA polymerase using a primer pair #382/383 and E. coli genomic DNA as a template. The insert was cloned into BamH I site of pEXoIN with Choo-Choo Cloning kit. The construct was named as pEXoIN-RecA, and the insert was confirmed by sequencing using oligonucleotide #376. The purified plasmid DNA was prepared using HiSpeed Plasmid Maxi kit (QIAGEN).

e. pEEP-RB1E7

Using primer pair #447/448 and oligonucleotide #446 as template the fragment which corresponds to RB1 exon7 region is amplified with KlenTaq DNA polymerase. The DNA fragment was inserted into Nde I site of pEEP with Choo-Choo Cloning kit. Primer pair #377/394 was used to screen positive clones by PCR with KlenTaq DNA polymerase. The construct was named pEEP-RB1E7, and the insert was confirmed by sequencing using oligonucleotide #377. The purified plasmid DNA was prepared using HiSpeed Plasmid Maxi kit (QIAGEN).

f. pEEP-CTCFE2

Using primer pair #452/453 and an oligonucleotide #451 as template, the fragment which corresponds to CTCF exon 2 region was amplified with KlenTaq DNA polymerase, and the DNA fragment was inserted into Nde I site of pEEP with Choo-Choo Cloning kit. Primer pair #377/394 was used to screen positive clones by PCR with KlenTaq DNA polymerase. The construct was named pEEP-CTCFE2, and the insert was confirmed by sequencing using oligonucleotide #377. The purified plasmid DNA was prepared using HiSpeed Plasmid Maxi kit (QIAGEN).

g. pEEP-mCTCF

Using primer pair #455/456 and an oligonucleotide #454 as template, the fragment which corresponds to mouse CTCF promoter region was amplified with KlenTaq DNA polymerase. The DNA fragment was inserted into Nde I site of pEEP with Choo-Choo Cloning kit. Primer pair #377/394 was used to screen positive clones by PCR with KlenTaq DNA polymerase. The construct was named pEEP-mCTCF, and the insert was confirmed by sequencing using oligonucleotide #377. The purified plasmid DNA as prepared using HiSpeed Plasmid Maxi kit (QIAGEN).

h. pEGFP-TP53E6

Using a primer pair #466/467 and an oligonucleotide #465 as template, the fragment which corresponds to human and mouse TP53 exon 6 region was amplified using KlenTaq DNA polymerase, and the DNA fragment was inserted at Ase I site of pEGFP with Choo-Choo Cloning kit. Primer pair #468/469 was used to screen positive clones by PCR with KlenTaq DNA polymerase. The construct was named pEGFP-TP53E6, the insert was confirmed by sequencing using oligonucleotide #468. The purified plasmid DNA was prepared using HiSpeed Plasmid Maxi kit (QIAGEN).

Inverse PCR.

Genomic DNA of pEEP-NF1E32N and pEEP-NF1E32NP-transfected GM00498 clones were extracted using Wizard SV Genomic DNA purification System (Promega). One μg of genomic DNA was digested with 10 unit of EcoR I (NEB Biolabs) in total of 50 μl volume at 37° C. for 6 hours, then denatured at 65° C. for 20 min. 50 μl EcoR I-cut DNA was mixed with 90 μl of 10×T4 DNA ligase buffer, 10 μl of 100 mM ATP and 400 unit of T4 DNA ligase (NEB Biolabs). Sterile Milli Q water was added to a total volume of 1 ml, and the mixture was incubated at 16° C. overnight. The DNA was precipitated in 1 ml of isopropanol and centrifuged at 12,000 rpm for 15 min. The supernatant is aspirated and washed in 1 ml of 70% ethanol. After centrifugation at 12,000 rpm for 10 min, the ethanol was aspirated and the precipitate was allowed to air dry for 5 min. The DNA pellet was then dissolved in 100 μl of sterile Milli Q water. For the 1^{st} round of PCR, 1 μl of the ligated DNA was mixed with 4 μl of 5×HF buffer, 1 μl of 100 mM dNTPs, 1 μl of a primer pair #435/437 (20 μM), 0.6 μl of DMSO, and 0.2 μl of Phusion DNA polymerase in a total of 20 μl reaction mixture. PCR was performed in My Cycler thermo cycler (Bio-Rad) in the following thermo cycles: 98° C. for 1 min, 25 cycles of 98° C. for 15 sec, 60° C. for 20 sec and 72° C. for 3 min, extension at 72° C. for 10 min, hold at 4° C. For the second round PCR, 1 μl of 100× diluted first PCR product was mixed with 4 μl of 5×HF buffer, 1 μl of 100 mM dNTPs, 1 μl of a primer pair #440/441 (20 uM), 0.6 μl of DMSO, and 0.2 μl of Phusion DNA polymerase in a total reaction mixture of 20 μl. PCR was performed as described above. Five μl of PCR product was used for running a 1% agarose gel.

### Example 2

### NF1 Gene Suppression

As shown in FIG. 3, linked LNA (#274/275) was designed to target the NF1 exon 32 region, linked LNA bound-pEEP-NF1E32N or pEEP-NF1E32NP was added to cultured human skin fibroblast GM00498 cells with pEXoIN-RecA and the same linked LNA for microporation; the stable cell clones, which were formed in less than 3 weeks, were selected by puromycin and EGFP fluorescence.

FIG. 3B shows the DNA sequence of ECR15 and linked LNA construction, SEQ ID NO: 153, 154, 155 and 156. The SEQ ID NO: 152 shows the DNA sequence of ECR15 is noted with an initial number and ending number on chromosome 17. Various evolutionary conserved regions ECR 20, ECR18, ECR4, ECR11 and ECR 15 are shown in relation to the transcripts. Each ECR is shown as a vertical bar which shows their location at the NF1 locus, respectively. The following sequence shows a chromosomal region in chromosome 17 that is targeted, beginning with Chr17:26,604,430 and ending with Chr17:26,604,545: atgaatgcaa agaaacttaa tttcaaggcc ttaggaaacg ctgactgtct tcattcttgc ttcttgtttg aa ggtaatgt gagtggtttc ttttcccaga gatgacagtg tttctt SEQ ID NO: 152 The targeting site of the linked LNA is underlined below. The CTCF binding motif is shown near the 3′ end of and is ttttcccaga gatgacagtg.

FIG. 3 B shows linked LNA construction illustrated as two 20-mer LNA oligonucleotide structure with a 7-mer linker, each LNA oligonucleotide is labeled (LNA1 and LNA2). Target DNA duplex is shown in lower case corresponding to the underlined region above. In the present linked LNA sequences, locked nucleic acids are shown in upper case, and normal nucleic acids are shown in lower case.

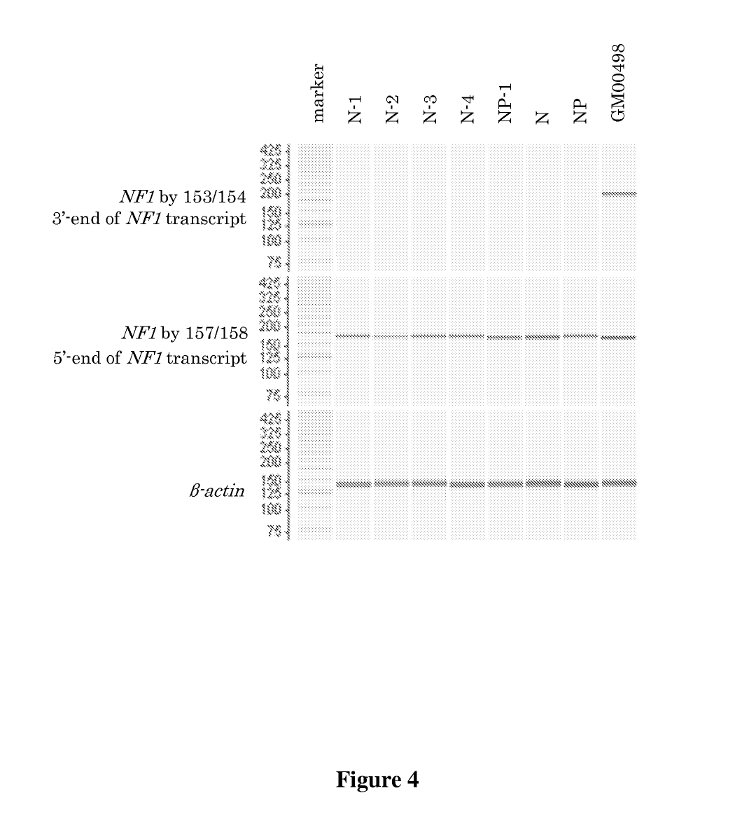

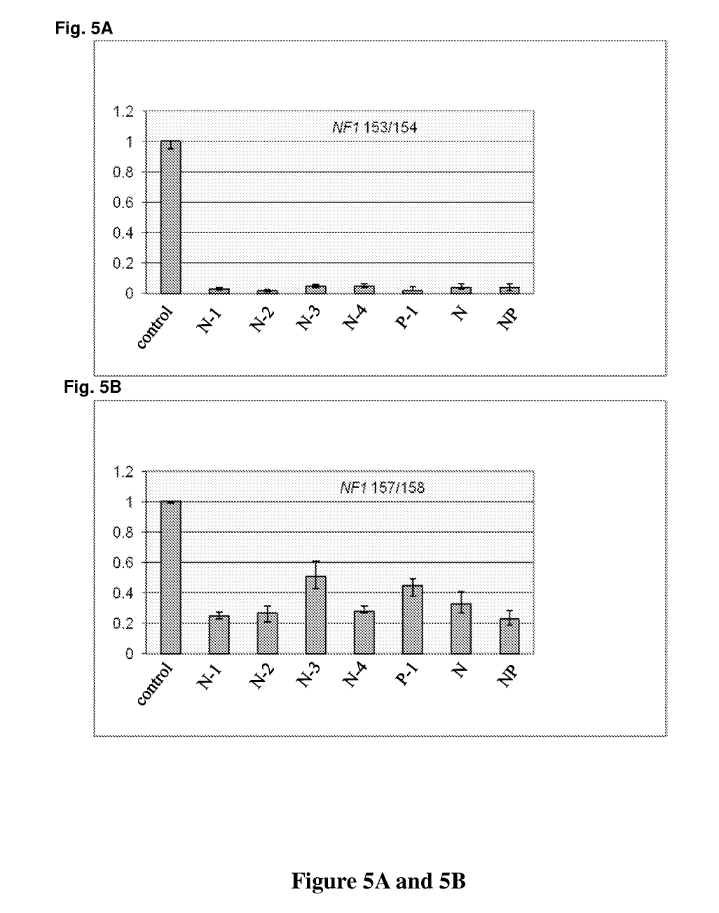

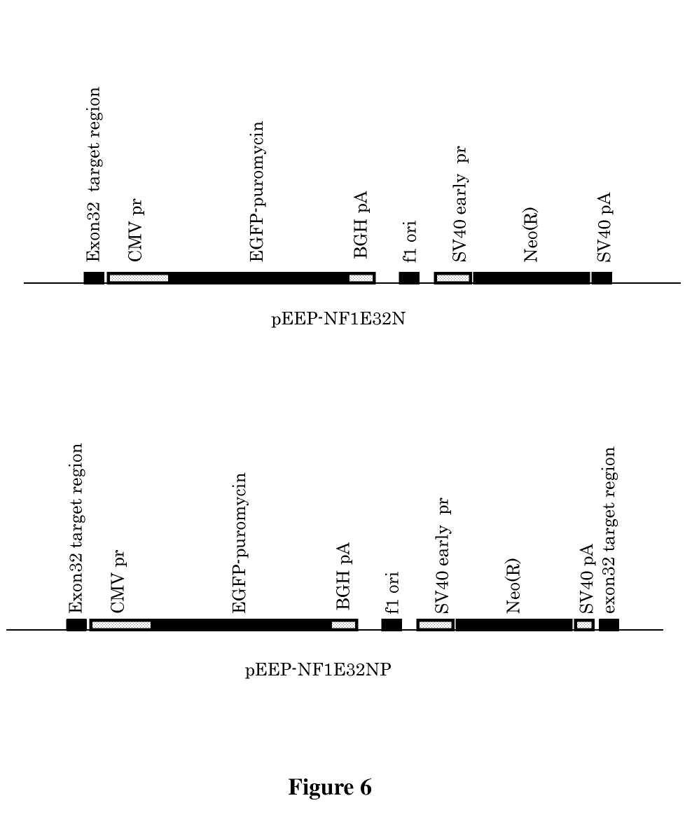

The recombination efficiency is 0.1%. NF1 expression in randomly selected stable cell clones was detected by RT-PCR and Q-PCR using primer pairs #153/154 and #157/158. β-actin served as a control, and was measured using primer pair #5899/5900 (FIGS. 4 and 5). NF1 expression was nearly 100% suppressed, as it cannot be detected by Q-PCR when examining the 3′-end of the transcript using primer pair #153/154. The quantity of the 5′-end transcript was also decreased, probably as a result of degradation due to the absence of the 3′-UTR of the transcript. Insertion or knock-in of the gene specific construct pEEP-NF1E32N or pEEP-NF1E32NP, which contains BGH and rgw SV40 polyA signal sequence (FIG. 6), may terminate NF1 transcription at the exon32 region, which was the recombination site. The recombination between NF1 exon 32 region and construct pEEP-NF1E32N or pEEP-NF1E32NP may employ the cell's innate repair machinery. linked LNA invasion of the exon32 region of the NF1 gene may induce DNA conformation change, which may bind and then be cut by RecA. DNA breakage formed at the locus may then activate the DNA repair machinery. At the same time, RecA can also bind with the construct pEEP-NF1E32N or pEEP-NF1E32NP at its single-strand DNA region which was invaded by the same linked LNA. The construct can be anchored at the broken DNA region due to formation of a RecA dimer locally, and this structure may facilitate efficient recombination at the locus. Suppression of NF1 expression of nearly 100% efficiency shows that the gene-specific construct was integrated into the site of both of alleles. It was also confirmed by inverse PCR. One band of the correct size was amplified by inverse PCR using vector specific nested primers in the pEEP-NE1E32N or pEEP-NF1E32NP integrated clones (N-1, 2, 3, 4 and NP-1, 2), which shows that the construct was integrated into the specific site, and not randomly in the genome.

In FIGS. 5A and 5B, clone N-1, 2, 3, 4 and N were selected from pEEP-NF1E32N transfected GM00498; Clone NP and NP-1 were selected from pEEP-NF1E32NP transfected GM00498. Clone N was mixed colonies after picking up single colony. Clone NP was also mixed colonies after picking up single colony. The control was normal GM00498 cell.

### Example 3

### CTCF Gene Suppression

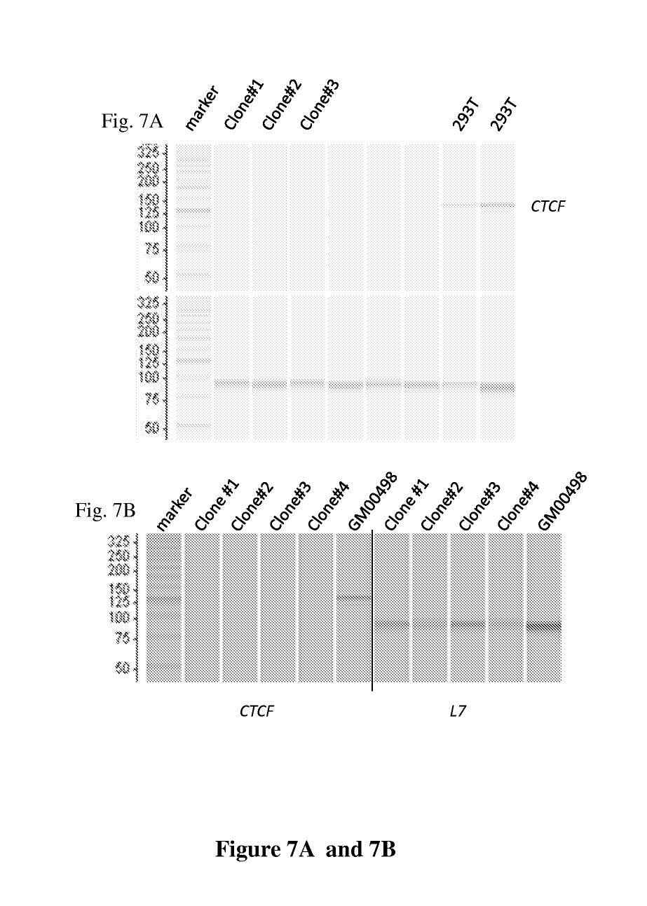

Linked LNA (#449/450) was designed to target to human CTCF exon 2 region. Linked LNA-bound pEEP-CTCFE2 was added into human skin fibroblast cell GM00498 or human renal cell 293T with pEXoIN-RecA and the same linked LNA for microporation. The stable cell clones, which formed in less than 3 weeks in GM00498 cells and less than 2 weeks in 293T cells, were selected by puromycin and EGFP fluorescence. The recombination efficiency is 0.12% in GM00498 cell and 30% in 293T cells. CTCF expression in randomly selected stable cell clones was measured by RT-PCR using primer pairs #3332/3333; L7 was served as control using primer pair #1266/1267. CTCF expression was suppressed by 100% both in the GM00498 clones (clone 1, 2, 3 and 4) and the 293T clones (clone 1, 2, 3, 4, 5 and 6) (FIG. 7). Cell morphology was altered in transfected 293T cells, which now appeared more like fibroblast cells after CTCF suppression. They attached to the plate much more tightly than did the original 293T cell line. This indicates that the locked nucleic acid-mediated gene regulation (LGR) technology can be used to manipulate cell lines by altering gene expression to make customized cell lines for research. For example, it will be possible to convert suspension B cell and T cell lines to adhesive cell lines, which may allow investigators to more easily pick stable colonies from transfected cells, and facilitate studies of immunology and cancer.

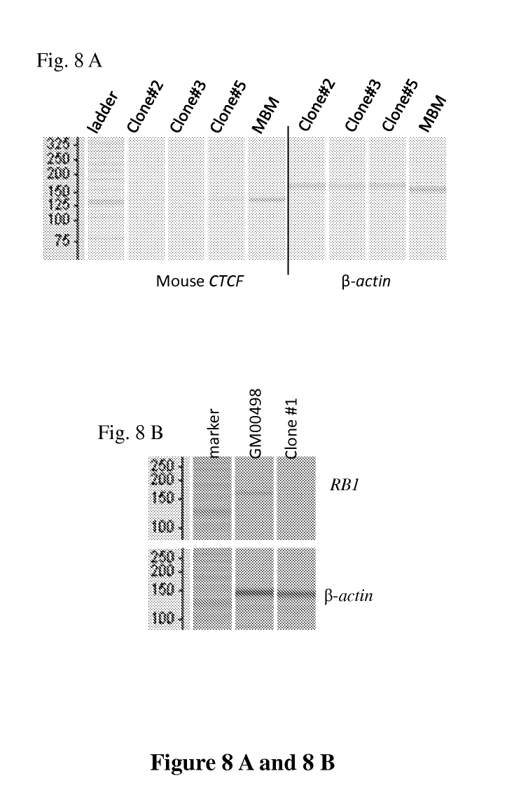

Linked LNA (#400/401) was designed to target the mouse CTCF promoter region. Linked LNA-bound pEEP-mCTCF was added to the mouse newborn fibroblast cell line MBM with pEXoIN-RecA and the same linked LNA by microporation. The stable cell clones, which were formed in less than 2 weeks in MBM cells, were selected by puromycin and EGFP fluorescence. The recombination efficiency was 23% in MBM cells. CTCF expression in randomly selected stable cell clones was detected by RT-PCR using primer #3328/3329; β-actin served as controls. CTCF expression was not 100% suppressed in the stable MBM clones (Clone 2, 3 and 5) (FIG. 8A). These results show that LGR technology can be used to fine-tune gene expression level or ablate expression entirely. By targeting to an exon region, varying levels of expression can be achieved, while targeting to the promoter region can decrease or abolish promoter activity. The ability to fine-tune gene expression will be extremely useful for the regulation of null-lethal or housekeeping genes.

### Example 4

### RB1 Gene Suppression

Linked LNA (#444/445) was designed to target to RB1 exon 7 region. Linked LNA-bound pEEP-RB1E7 was added to human skin fibroblast cell GM00498 with pEXoIN-RecA and the same linked LNA by microporation. The stable cell clones, which were formed in less than 3 weeks, were selected by puromycin and EGFP fluorescence. The recombination efficiency was 0.07%. RB1 expression in the selected stable cell clones was detected by RT-PCR using primer #472/473; β-actin served control (FIG. 8B).

### Example 5

### Double Suppression of TP53 and NF1 in One Cell Line

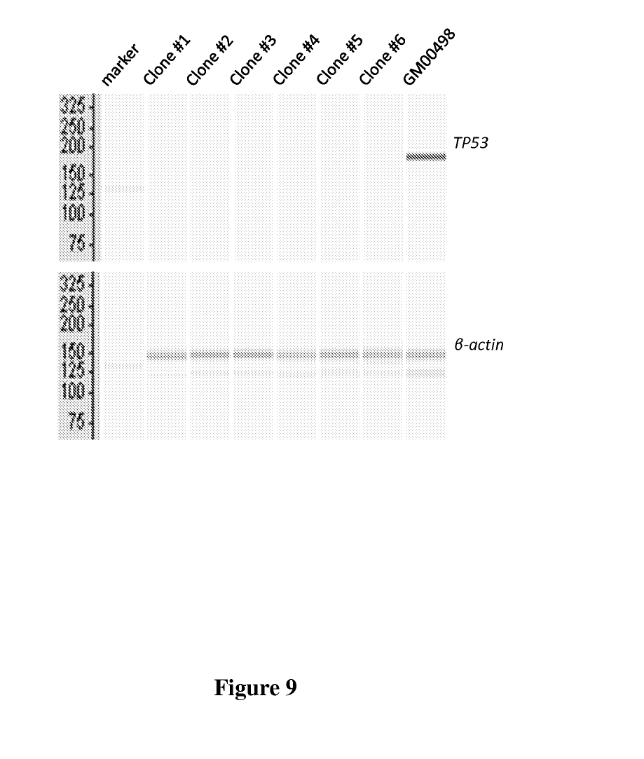

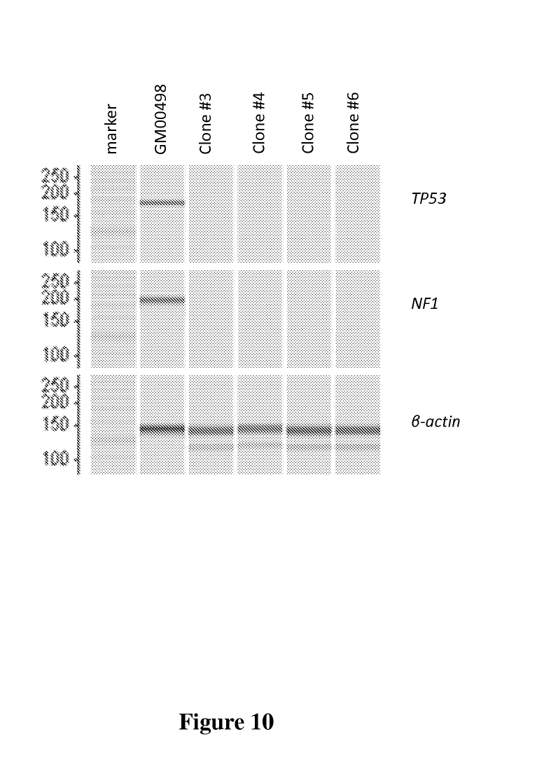

Linked LNA (#463/464) was designed to target to human TP53 exon 6 region. Linked LNA-bound pEGFP-TP53E6 was added into human skin fibroblast cell GM00498 with pEXoIN-RecA and the same linked LNA by microporation. The stable cell clones, which were selected by G418 and EGFP fluorescence, were formed in less than 3 weeks. The recombination efficiency was 2.9%. TP53 expression in the selected stable cell clones were detected by RT-PCR using primer #478/479; β-actin served as control (FIG. 9). Linked LNA-bound pEEP-NF1E32N and its linked LNA (#274/275) were added into pEGFP-TP53E6 transfected stable clone #1 with pEXoIN-RecA by microporation. The TP53⁻INF1⁻ stable clones, which were selected by puromycin, G418 and EGFP fluorescence, were formed in less than two weeks. The recombination efficiency is 5.7%. Expression of TP53 and NF1 was detected by RT-PCR using the primer pairs described above; β-actin served as control (FIG. 10). These results show that LGR technology can be used to make a stable cell line with 2 or more gene mutations. Such cell lines will be very valuable for the study of gene-gene interactions and cell signaling in complex disease development. It is probably the first report to make multiple mutations in one cell in less than 2 months.

### Examples 6-8 pertain to Long Range Gene Modulation

### Example 6

### Materials and Methods

Cell Culture.

Fibroblast cell lines GM00010, GM00622, GM016933, GM01639, GM01858 and GM01859 were purchased from CORIELL Institute for Medical Research, and were cultured in minimum essential medium (MEM) supplemented with 1× non-essential amino acids (NEAA) and 15% fetal bovine serum (Invitrogen). Five μg/ml of Plasmosin (InvivoGen) was also added to the culture medium to protect the cells from mycoplasma contamination. HeLa cells (ATCC) and a primary fibroblast cell HFB1, derived from human skin tissue in our laboratory, were cultured in Dulbecco's modified Eagle's medium (DMEM) (Invitrogen) supplemented with 10% fetal bovine serum and 5 μg/ml of Plasmocin. All cell lines were grown at 37° C. with 5% of CO₂.

Cell Transfection.

LNA oligonucleotides were synthesized by Biosynthesis Inc, and dissolved in sterile Milli Q water at 100 μM stock concentration. Linked LNA was prepared as described above. Cells for microporation were prepared following the protocol provided by the vendor. Trypsinized GM01859 cells were collected, counted and centrifuged at 1,200 rpm for 5 minutes at room temperature, and the cells were resuspended in 2 ml of 1×DPBS (Mediatech Inc). 1.5×10⁶ cells were transferred into two microtubes and spun again for 2 minutes under the same conditions. The cells were resuspended in 300 μl of resuspension solution provided by Digital Bio Technology after removing the DPBS in each tube. Nine μM linked LNA was mixed with the cells by gentle pipetting. Microporation was performed in a 100-μl tip at 1,600 volt for 20 milliseconds in a MicrioPorator MP-100 (Invitrogen). Three independent microporations were performed for the cells with or without linked LNA, and the transfected cells were incubated in a 6-well plate with 2 ml of medium described above without Plasmosin for 48 hours before harvesting. One μg of purified pEGFP was used as an indicator for assessing the transfection efficiency of each microporation.

Quantification of NF1 Expression by Real Time-PCR.

LNA 1 and LNA2 were purchased from Biosynthesis Inc. linked LNA-treated and untreated GM01859 cells were lysed in 1 ml of Tri Reagent (Sigma-Aldrich) after removing the culture medium. Total RNA was purified by following the protocol provided by the vendor. One μg of total RNA was used to synthesize cDNA using SuperScript III First-Stand Synthesis SuperMix kit (Invitrogen) following the protocol provided by the vendor. Allele-specific primers were used to amplify the NF1 gene at its 5′-end and 3′-end. Primer pairs #153/#307 and #153/#308 were used for amplifying the transcripts containing the SNP rs1801052 with an Rsa I recognition site; the primer pairs #305/#158 and #306/#158 were used to amplify the transcripts containing the SNP rs2040793 with a Kpn I recognition site. The following thermo cycle for RT-PCR with KlenTaq DNA polymerase (Ab Peptides Inc.) and ³²P-dCTP (Perkin Elmer) mixed dNTP was used: 95° C. 2 min, 36 cycles of 95° C. 20 sec, 63° C. 30 sec and 72° C. 20 sec, followed by 5 min extension at 72° C. PCR products were subjected to digestion by Rsa I or Kpn I (NEB Biolabs), respectively, and were analyzed on a 5% polyacrylamide-urea gel and visualized with a PhosphoImager. Real time-PCR assays for allele-specific expression of NF1 were run in quadruplicate in a 384-well plate using an ABI Prism 7900HT, SYBR Green and the ABI protocol with the four primer pairs described above. Relative levels were determined by a “delta Ct and delta-delta Ct” with reference to a human β-actin control using primer pairs #5899 (SEQ ID No: 56) and #5900 (SEQ ID No: 57). All of the primers are listed in Table 3.

TABLE 3
Oligonucleotides used in Example 6

| | | SEQ | |
|---|---|---|---|
| Oligo | | ID | |
| # | Sequence | No: | Memo |
| 3959 | GATACTTTGTAGGAGCTGAGCACAG | 60 | ECR1 PCR primers |
| 3960 | CTGAGTGAAACCTTGTTCTAAGCTG | 61 | |
| 3961 | CCCAGAGGAGTTAGATGACGTCAC | 62 | ECR2 PCR primers |
| 3962 | CACAGTCCGAGACGCCGCCATGAC | 63 | |
| 3963 | CACAGGCCGGTGGAATGGGTCCA | 64 | ECR3 PCR primers |
| 3964 | CTACCTCCCCTCACCTACTCTGTC | 65 | |
| 3965 | CCTTGTCCCTGACCAGCCTCCGAC | 66 | ECR4 PCR primers |
| 3966 | CTAAAGGCGCCCCCAGGTCACTCATC | 67 | |
| 3967 | AGGGACAGGAGGTATCGGAAGGCTC | 68 | ECR5 PCR primers |
| 3968 | CCTTTTAACAGTTTAAGCTGACCAC | 69 | |
| 3969 | GAAAATGAGTAGAATGTACACAGAG | 70 | ECR7 PCR primers |
| 3970 | GTGAACAATGGTTAATTTGTTTCTAG | 71 | |
| 3971 | CTAAATAGCTGAAGCTTCTGGCTTGA | 72 | ECR6 PCR primers |
| 3972 | TGGCAAAGATTCTATGCGCTAGTT | 73 | |
| 3973 | CATAGAGCACTTTCAAGCATGGACT | 74 | ECR8 PCR primers |
| 3974 | CAGCCACTTTCACCAAGTACACTG | 75 | |
| 3975 | GGCACTAAAAACTAGACATCAGAG | 76 | ECR9 PCR primers |
| 3976 | CTGATCCTAGTGAAATATTTTTTAAAC | 77 | |
| 3977 | GTATGACCCATTGTAACTACATCAG | 78 | ECR10 PCR primers |
| 3978 | GATAAAACCAACAGGTGAAGACATG | 79 | |
| 3979 | CCAGTTCTAGAATCATAGCTCTGA | 80 | ECR11 PCR primers |
| 3980 | AACTGTTTCCCTTTTGCTTCTTGATAG | 81 | |
| 3981 | CGGCTTCAGTTGCTTAGAGACGT | 82 | ECR12 PCR primers |
| 3982 | AGATACATCTTTAAAAATAGCAATGGTG | 83 | |
| 3983 | GTCAATTGAAGGATACACAGAGAAG | 84 | ECR13 PCR primers |
| 3984 | TTCATTACAAGTAGATCACACACT | 85 | |
| 3985 | GAAATAGTAGACATGATTGGGTCTCA | 86 | ECR14a PCR primers |
| 3986 | GCAACAAAAACAATGTTGGCTTGTAC | 87 | |
| 3987 | CACTTCAGCCTATTTGACCTTCACTG | 88 | ECR14b PCR primers |
| 3988 | AGGAACACAGAGAATGTTTCAATG | 89 | |
| 3989 | TTTCAAGGCCTTAGGAAACGCTGAC | 90 | ECR15 PCR primers |
| 3990 | AAGAAACACTGTCATCTCTGGGA | 91 | |
| 3991 | AATGCTTCAGCCTTCTAATTCTCAG | 92 | ECR16 PCR primers |
| 3992 | GCATTAGTATAACACTGAACAAGAGTG | 93 | |
| 3993 | ACAGAGTCTCATCTCCGCAGGTGCA | 94 | ECR17a PCR primers |
| 3994 | TCTGCATCGAAGCCTGACTGAAGTC | 95 | |
| 3995 | TGCTGGGTGACATTGGATGCTGAG | 96 | ECR17b PCR primers |
| 3996 | GGTTGAACTTTCTCCAACCATGCAG | 97 | |
| 3997 | GTCAACCTTCTCAGGTGGCTCTGA | 98 | ECR17c PCR primers |
| 3998 | CCTAAGGAGGTTGAACCATCTGCA | 99 | |
| 3999 | TAATTTTCAGGTCTATTTAGCTGCTGA | 100 | ECR17d PCR primers |
| 4000 | GGTGAGTCTTCTCCAATCCAGCAG | 101 | |
| 4001 | AAGATTCAACCTCTCCAGAAGACTC | 102 | ECR17e PCR primers |
| 4002 | TCCAGAGGAGGTGGCACCTTCTGCA | 103 | |
| 4003 | CTGGATGTCTTCAAGGGTCTCTGGA | 104 | ECR17f PCR primers |
| 4004 | ACTGTATTCCGGCAGCCTGCCTCCAG | 105 | |
| 4005 | GAATATACTCACAGTTTCTCGGTCCA | 106 | ECR18 PCR primers |
| 4006 | TTCTTCACAGAAATTTCCAAGGAAAC | 107 | |
| 4007 | GATTAACTAACTTACAAATACTGGA | 108 | ECR19 PCR primers |
| 4008 | TAACTTTTTCATTTTCCAGAATTCTCAGT | 109 | |
| | G | | |
| 4009 | CTTCTATTTCACTTACAAGTTGTG | 110 | ECR20 PCR primers |
| 4010 | TGTCTTTTTTTGTAGAAATCTGGGCTGCA | 111 | |
| 4011 | TGCATGGCACCAAGAATGTTACCA | 112 | PCR primers for WSB1 |
| 3952 | GACTCCAGACTATATCTCCACAGTC | 113 | exon3 |
| 4012 | ACTGGGGGCTCTTGCGTAGCACATG | 114 | PCR primers for CTCF |
| 4013 | GGCTGTGATGTGTGAGCCTGCACTG | 115 | binding site 3 |
| 4018 | GGAATTGGTTGTAGTTGTGGAATCG | 116 | PCR primers for CTCF |
| 4019 | GATGACCCCCGTGAACCCTGCGAC | 117 | binding site 6 |
| 153 | CTAAAGAAGGTTGCGCAGTTAGCAG | 118 | Primers for Q-PCR of |
| 307 | CATATCAGTCTGTGGGATCTGGTAC | 119 | 5′-transcript of NF1 |
| 308 | CCATATCAGTCTGTGGGATCTGGTAT | 120 | |
| 158 | ACTTCCTCTGGACTCTTGTCATTG | 121 | Primers for Q-PCR of |
| 305 | GGTCAACTTGTATTCAGCAGGTACC | 122 | 3′-end transcript of |
| 306 | GGTCAACTTGTATTCAGGAGGTACG | 123 | NF1 |
| 331 | TTGCCATCTTTAGACATGCACATG | 124 | Primers for 3C PCR of |
| 335 | ACATAAAGTTCATTGTTACTAGCATCATC | 125 | ECR15/ECR4 and |
| 337 | GAATACAGTGTTCATCTGGTTTCATC | 126 | ECR15/ECR11 |
| 359 | TCTGGCCTCGGCGGCCAAGCTTTTGTTGC | 127 | Primers for cloning of |
| | ATTGGATATCCTTGGTC | | ECR15 region into |
| 360 | ATACCCTCTAGTGTCTAAGCTTCTAAATT | 128 | pGL4.23[luc2/minP |
| | ATGCCTGAGACTGCCTAG | | |

Chromatin-immunoprecipitation (ChIP) Assay.

The ChIP assay was performed as previously described (Ling, J. Q., et al., “CTCF mediates interchromosomal colocalization between Igf2/H19 and Wsb1/Nf1” Science, 2006. 312(5771): p. 269-72). Approximately 50,000 cells were fixed with 1% formaldehyde, and were then sonicated for 300 sec (20 sec on and 40 sec off) in an ice bath using a Branson sonicator with a 2-mm microtip and a setting of 40% for output control and 90% for duty cycle. The sonicated chromatin (0.6 ml) was clarified by centrifugation, aliquoted and snap-frozen in liquid nitrogen. Sonicated chromatin (20 μl) was diluted ten-fold, cleared with salmon sperm DNA/protein A-agarose (80 μl), and purified with mouse anti-CTCF MAb (10 μl, BD Biosciences Pharmingen) or mouse anti-RNA polymerase II MAb (Upstates), and Protein A-agarose (60 μl). The DNA was extracted from bound chromatin after reversal of cross-linking and proteinase K treatment, and was further purified using a QIAGEN PCR purification kit, and then eluted in 100 μl of sterile distilled water. ChIP PCR reactions (4.5 μl under liquid wax) contained 1.5 μl ChIP (or input) DNA, 0.1 μM appropriate primer pairs, 50 μM dNTP mixture with ³²P-dCTP (Perkin Elmer), and 0.2 units KlenTap I. Primer Pair #3965/#3966 is for PCR of ECR4, primer pair #3979/3980 was for ECR11, primer pair #3989/#3900 was for ECR15, primer pair #3999/#4000 was for ECR17, primer pair #4005/4006 was for ECR18, primer pair #4009/#4010 was for ECR20, and primer pair #4012/#4013 was for CTCF binding site #3 of ICR at the IGF2/H19 locus. Standard PCR conditions were the same as described above for RT-PCR of NF1 expression in 32 PCR cycles. ChIP PCR products were separated and analyzed on a 5% polyacrylamide-urea gel using the PhosphoImager. Semi-quantification of each PCR products was performed in ImageQuant software (Invitrogen). All primers used in the experiment are listed in Table 3.

Chromosome Conformation Capture (3C) Assay.

3C assays were carried out essentially as described (www.epigenome-noe.net/researchtools/protocol) with modifications. Cells were fixed in 2% formaldehyde, quenched with 0.125 M glycine, and then lysed with 0.2% NP-40 on ice for 2 h with stirring. Nuclei were collected by centrifugation, resuspended in 1×NEB buffer 2 (New England Biolabs) containing 0.3% SDS, treated at 37° C. for 1 h and quenched with 1.8% Triton X-100 at 37° C. for 1 hour. Treated nuclei (0.5 million) were digested with 1000 units of Hind III in 500 μl of 1×NEB buffer 2 at 37° C. for ˜16 h. After enzyme deactivation in 1% SDS at 65° C. for 20 min and quenching in 2.6% Triton X-100 at 37° C. for 1 hour, aliquots of digested nuclei (˜2 μg DNA) were ligated with T4 DNA ligase (NEB Biolabs); two thousand units of T4 DNA ligase were used in a 1 ml ligation reaction at 16° C. for 4 h. Cross-linked DNAs were treated with Proteinase K at 65° C. overnight, followed by RNase A digestion at 37° C. for 30 minute, and concentrated in a speedVac (Savant) to reduce to ˜⅓ volume before phenol-chloroform extraction. DNA samples were precipitated with ammonium acetate (2M) and 2-propanol (1.5× volume). The pellet was washed with cold 75% ethanol three times and then dissolved in sterile Milli Q water. Hind III recognition sites at chr17:26,450,184 close to ECR4, chr17:26,574,478 close to ECR11 and chr17:26,605,119 close to ECR15 were chosen in this 3C experiment. Primers for 3C were designed near these sites. BAC clone RP11-1107G21 DNA was purchased from Roswell Park Cancer Institute. Two μg of the BAC clone DNA were digested by Hind III overnight at 37° C. in 50 μl volume followed by denaturing at 65° C. for 20 minutes and cooling to 15° C. One mM ATP and 2,000 units of T4 DNA ligase were added to the digested DNA solution and incubated at 15° C. overnight. One hundred ng of the religated BAC clone DNA served as control, and 3C-ligated genomic DNA was used for PCR with KlenTaq DNA polymerase and dNTPs containing ³²P-dCTP in the following thermo cycle: 95° C. 2 min, 36 cycles of 95° C. 20 sec, 57° C. 30 sec and 72° C. 30 sec, followed by 5 min extension at 72° C. PCR products were separated and analyzed on a 5% polyacrylamide-urea gel using the PhosphoImager. Primer pairs #331/J#337 and #335/#337 were used to amplify the ECR15/ECR4 interaction and the ECR15/ECR11 interaction, respectively. All primers used in the experiment are listed in Table 3.

In Vitro Binding of Linked LNA and ECR15.

The ECR15 fragment was amplified by primer pair #3989/#3990 in KlenTaq DNA polymerase and BAC clone RP11-1107G21 DNA using the following thermo cycles: 95° C. 2 min, 36 cycles of 95° C. 20 sec, 63° C. 30 sec, 72° C. 20 sec, followed by 5 min extension at 72° C. Linked LNA was prepared as described above. One μM of linked LNA was mixed with 200 ng of ECR15 PCR products in 50 mM phosphate buffer, pH 7.4, and incubated at 37° C. overnight. The ECR15 and linked LNA-bound ECR15 PCR products were run on a 2% agarose gel for 30 min. Gel image was acquired by ChemiImager 4400 (AlphaInnotech).

Dual-Luciferase Reporter (DLR) Assay.

ECR15 region of NF1 was amplified by Phusion DNA polymerase (NEB Biolabs) with primer pair #359/#360 and BAC clone RP11-1107G21 as template in the following thermo cycles: 98° C., 1 min, 5 cycles of 98° C. 15 sec, 57° C. 30 sec and 72° C. 10 sec, 20 cycles of 98° C. 15 sec, 65° C. 30 sec, and 72° C. 10 sec, followed by 10 min extension at 72° C., then hold at 4° C. The ECR15 PCR products were cloned into pGL4.23[luc2/minP] (Promega) cut by Hind III (NEB Biolabs) using the protocol of Choo-Choo Cloning kit. Positive clones of pGL4.23-ECR15 were confirmed by sequencing. All of the plasmids, pRL-TK, pGL4.23[luc2/minP] and pGL4.23-ECR15, were prepared with QIAfilter Plasmid Midi Kit (QIAGEN). Renilla luciferase expression vector pRL-TK served as an internal control in the DLR assay. Fifty ng of pGL4.23[luc2/minP] and pGL4.23-ECR15 were mixed with 5 ng of pRL-TK, respectively, and transfected into Hela cells with FuGENE HD Transfection reagent (Roche Applied Science) following the protocol provided by the vendor. The transfected Hela cells were cultured in a 96-well plate for 48 hour before DLR assay. Four repeats were performed for the experiment. Luciferase activity was analyzed with Dual-Luciferase Reporter Assay System (Promega) following the vendor's protocol with a minor modification. Briefly, the transfected cells were washed in 100 μl 1×PBS after 48 hour culture, and lysed in 50 μl of 1× passive lysis buffer for 15 min at room temperature on a rocking platform. Twenty μl of the cell lysate were transferred into a luminometer tube containing 100 μl of LAR II and mixed by pipetting 3 times. Luminescence was detected by 20/20″ Luminometer (Turner Biosystems). 100 μl of Stop & Glo reagent was then transferred into each tube and vortexed briefly to mix. Renilla luciferase activity in each reaction was also detected by the same Luminometer. The relative activity of firefly luciferase was normalized with Renilla luciferase activity in each corresponding reaction. The luciferase activity in the pGL4.23-ECR15 transfected cells was compared with the control cells transfected with pGL4.23[luc2/minP], and the average value from the four repeat assays was presented.

### Example 7

### CTCF Binding Sites at NF1 Locus

CTCF is required for many long-range chromatin interactions. Ling, J. Q., et al. previously demonstrated that CTCF bound to several DNA regions near the Nf1 gene on mouse chromosome 11 (“CTCF mediates interchromosomal colocalization between Igf2/H19 and Wsb1/Nf” Science, 2006. 312(5771): p. 269-72). To compare the mouse and human NF1 genes, we used Genome Browser (http slash slash colon www(dot)dcode.org) to search for evolutionary conserved regions (ECR) between the 204 kb Wsb1-Nf1 intergenic region on mouse chromosome 7 and human chromosome 17. Twenty ECRs located in intergenic and intronic regions near the human NF1 locus were identified based on 85% minimum identity per 100-bp sequence (Table 4).

TABLE 4
Evolutionary conserved regions (ECR) of human NF1 locus

| ECR | Position on chromosome 17 |
|---|---|
| ECR1 | 26,403,942-26,404,099 |
| ECR2 | 26,445,885-26,446,202 |
| ECR3 | 26,446,443-26,446,599 |
| ECR4 | 26,446,724-26,446,872 |
| ECR5 | 26,455,481-26,455,691 |
| ECR6 | 26,474,137-26,474,236 |
| ECR7 | 26,493,149-26,493,262 |
| ECR8 | 26,554,161-26,554,375 |
| ECR9 | 26,556,605-26,556,772 |
| ECR10 | 26,558,291-26,558,493 |
| ECR11 | 26,575,143-26,575,249 |
| ECR12 | 26,601,130-26,601,242 |
| ECR13 | 26,603,620-26,603,776 |
| ECR14 | 26,603,798-26,604,394 |
| ECR15 | 26,604,430-26,604,545 |
| ECR16 | 26,605,604-26,605,827 |
| ECR17 | 26,398,386-26,401,459 |
| ECR18 | 26,396,290-26,396,435 |
| ECR19 | 26,393,322-26,393,433 |
| ECR20 | 26,389,702-26,389,829 |

Note:
1. Detection of ECRs: minimum length is 100 bp and minimum identity is 85%
2. Human NF1is located at chr17: 26,446,071-26,728,821 29 based on human genome Assembly March, 2006 at UCSC browser.

We performed chromatin immunoprecipitation (ChIP) assays using an anti-CTCF antibody to detect CTCF binding sites at the human NF1 locus on chromosome 17q11.2 in a human fibroblast cell line, HFB1 (FIG. 11); similar studies were performed in another fibroblast cell line GM00010, the WTCL tumor cell line and the REH leukemia cell line (data not shown). Multiple CTCF binding sites were identified in and around NF1, including ECR17, ECR18 and ECR20, ˜50-57 kb upstream of the NF1 gene, ECR4 at the NF1 transcription initiation site near exon 1, and ECR11 and ECR15, ˜130-158 kb downstream from the transcription initiation site (FIG. 11). In genome-wide studies, three CTCF binding sites, INSUL_ZHA016436, INSUL_ZHA016437 and INSUL_ZHA016438, have been reported in the NF1 region, but, none of them is located at the same site as the CTCF binding sites we identified by ChIP assay in this study. When we screened these ECR regions using In Silico CTCFBS Prediction Tool (http://insulatordb.utmem.edu/), motifs that are characteristic of CTCF binding (REN_{—}20, MIT_LM2, MIT_LM7 and MIT_LM23) were identified in ECR4, ECR11, ECR15 and ECR17, respectively, with position weight matrices (PWM) scores more than 3 (Table 5); a sequence with a PWM score>3 is suggestive of a match.

TABLE 5
CTCF binding motifs in ECR regions at NF1 locus

| | | SEQ | Input | Motif | | Motif | |
|---|---|---|---|---|---|---|---|
| Motif | | ID | Sequence | Start | Motif | Orien- | |
| PWM | Motif Sequence | No. | Name | Location | Length | tation | Score |
| REN_20 | ATTAAATCCAGATGGCG | 129 | ECR4 | 118 | 20 | − | −0.117778 |
| | CGC | | | | | | |
| MIT_LM2 | TTAAATCCAGATGGCGC | 130 | ECR4 | 118 | 19 | − | −3.82585 |
| | GC | | | | | | |
| MIT_LM7 | TTAAATCCAGATGGCGC | 131 | ECR4 | 117 | 20 | − | 4.07071 |
| | GCC | | | | | | |
| MIT_LM23 | TTAAATCCAGATGGCGC | 132 | ECR4 | 117 | 20 | − | 2.29218 |
| | GCC | | | | | | |
| REN_20 | GGAAACAGTTGAGGGT | 133 | ECR11 | 97 | 20 | + | −9.55537 |
| | CCTC | | | | | | |
| MIT_LM2 | GAAACAGTTGAGGGTCC | 134 | ECR11 | 98 | 19 | + | −1.59254 |
| | TC | | | | | | |
| MIT_LM7 | GAAACAGTTGAGGGTCC | 135 | ECR11 | 98 | 20 | + | 4.19766 |
| | TCA | | | | | | |
| MIT_LM23 | TAGCCATGAGATCTAGG | 136 | ECR11 | 41 | 20 | − | −5.06617 |
| | TCT | | | | | | |
| REN_20 | TCCACAAATAGATGGCA | 137 | ECR15 | 651 | 20 | − | −2.28827 |
| | TCT | | | | | | |
| MIT_LM2 | TTTCCCAGAGATGACAG | 138 | ECR15 | 2091 | 19 | + | 3.30138 |
| | TG | | | | | | |
| MIT_LM7 | TCCCGAGTAGCTGGGAC | 139 | ECR15 | 2863 | 20 | − | 7.82876 |
| | TAC | | | | | | |
| MIT_LM23 | ACACCACCACGCCTGGC | 140 | ECR15 | 2837 | 20 | − | 2.85834 |
| | TAA | | | | | | |
| REN_20 | TCTACCTCCTGAGGGAA | 141 | ECR17 | 1136 | 20 | + | 9.8243 |
| | CCC | | | | | | |
| MIT_LM2 | GACCCAGCAGATGGCCC | 142 | ECR17 | 1168 | 19 | − | 12.6439 |
| | CA | | | | | | |
| MIT_LM7 | GACCCAGCAGATGGCCC | 143 | ECR17 | 1167 | 20 | − | 17.6811 |
| | CAT | | | | | | |
| MIT_LM23 | TAACCTGAAGGTGAAAC | 144 | ECR17 | 2275 | 20 | + | 7.88512 |
| | TGG | | | | | | |
| REN_20 | ATGGGCTGGGGGTGGTG | 145 | ECR18 | 524 | 20 | + | −1.95444 |
| | GCT | | | | | | |
| MIT_LM2 | GTTTCAACAGAGGGCAA | 146 | ECR18 | 1554 | 19 | − | 8.52602 |
| | AG | | | | | | |
| MIT_LM7 | GTTTCAACAGAGGGCAA | 147 | ECR18 | 1553 | 20 | − | 7.70739 |
| | AGC | | | | | | |
| MIT_LM23 | TGGGCTGGGGGTGGTGG | 148 | ECR18 | 525 | 20 | + | −0.964643 |
| | CTC | | | | | | |
| REN_20 | CTGCCCACCAGATGCCA | 149 | ECR20 | 1444 | 20 | + | 5.27707 |
| | GGA | | | | | | |
| MIT_LM2 | TGCCCACCAGATGCCAG | 150 | ECR20 | 1445 | 19 | + | 17.1601 |
| | GA | | | | | | |
| MIT_LM7 | TTCCCAGGAGGTGCTGG | 151 | ECR20 | 2539 | 20 | − | 13.0536 |
| | TCT | | | | | | |
| MIT_LM23 | TTCCCAGGAGGTGCTGG | 151 | ECR20 | 2539 | 20 | − | 7.30759 |
| | TCT | | | | | | |

Notes:
1. Searching of CTCF binding motif in the ECR4, ECR11, ECR15, ECR17, ECR18 and ECR20 regions was performed by a web-based tool: in silico CTCFBS Prediction Tool at http://insulatordb.utmem.edu/storm.php.
2. Input DNA sequence for the prediction was used their ECR core regions listed in Table S1 for ECR4, ECR11 and ECR17, and DNA sequences including their upstream and downstream 1-2 kb flank regions for ECR15, ECR18 and ECR20.
3. The matched CTCF binding motifs in these ECR regions were highlighted in yellow. A sequence with a PWM score more than 3 is suggestive of a match.
4. PWM, position weight matrices. +, plus strand of DNA duplex. −, minus strand of DNA duplex.
5. CTCF binding motif REN_20 was identified by Kim et al in 2007 (Cell, 2007, 128(6): p. 1231-45). CTCF binding motifs MIT_LM2, MIT_LM7 and MIT_LM23 were proposed by Xie et al in 2007 (Proc Natl Acad Sci U S A, 2007. 104(17): p. 7145-50).

In ECR4, a DNA sequence TTAAATCCAGATG GCGCGCC (SEQ ID No: 131) was matched to MIT_LM7 with a PWM score 4.07. In ECR11, DNA sequence GAAACAGTTGAGGGTCCTCA (SEQ ID No: 135) matched MIT-LM7 with a PWM score 4.85. In ECR15, DNA sequence TTTCCCAGAGA TGACAGTG (SEQ ID No: 138) matched MIT_LM2 with a PWM score 3.30. In ECR17, DNA sequence TCTACCTCCTGAGGGAACCC (SEQ ID No: 141) matched REN_{—}20 with a PWM score 9.82; DNA sequence GACCCAGCAGATGGCCCCA (SEQ ID No: 142) in its minus DNA strand matched MIT_LM2 with a PWM score 12.64, and SEQ ID No: 143 matched MIT_LM7 with a PWM score 17.68; and DNA sequence TAACCTGAAGGTGAAACTGG (SEQ ID No: 144) matched MIT_LM23 with a PWM score 7.88. Although there was no sequence suggesting a CTCF binding motif in the ECR18 and ECR20 core sequences, DNA sequence GTTTCAACAGAGGGCAAAG (SEQ ID No: 146) located ˜400 bp downstream of ECR18 in its minus DNA strand matched MIT_LM2 with a PWM score 8.53 and SEQ ID No: 147 matched MIT_LM7 with a PWM score 7.71. DNA sequence TGCCCACCAGATGC CAGGA (SEQ ID No: 150) located ˜300 bp downstream of ECR20 matched MIT_LM2 with a PWM score 17.16; DNA sequence TTCCCAGGAGGTGCTGGTCT (SEQ ID No: 151) located ˜1.4 kb downstream of ECR20 in its minus DNA strand matched MIT_LM7 with a PWM score 13.05, and also matched MIT_LM23 with a PWM score 7.31. DNA sequence TCCCGAGTAGCTGGGACTAC (SEQ ID No: 139) ˜700 bp downstream of ECR15 in its minus strand matched MIT_LM7 with a PWM score 7.83 (Table 5). These results are consistent with our CTCF-ChIP assay at the NF1 locus in several different cell types.

### Example 8

### Linked LNA Targeting to ECR15 Region Down-Regulates NF1 Expression

NF1 is a large gene covering a 282 kb DNA region, and it has 7 different transcripts that use combinations of various exons (according to the UCSC genome browser). The NF1 gene itself, located at 17q11.2, encompasses >300 kb of human chromosome 17. The 60 exons which constitute the human NF1 gene give rise to several alternatively spliced transcripts.

ECR15 was chosen as our target site for the linked LNA because it is located in the middle of the NF1 gene, and it has ideal DNA sequence specificity, thereby minimizing off-target effects. In order to assure that linked LNA would interact with the DNA without disturbing the CTCF binding at the binding site, the linked LNA target site was chosen so that it would be close to the CTCF binding motif in the ECR15 region but not within the motif site itself (FIG. 3B-C). Using this specific site, we hoped to avoid targeting any other MIT_LM2 CTCF binding motifs in the genome.

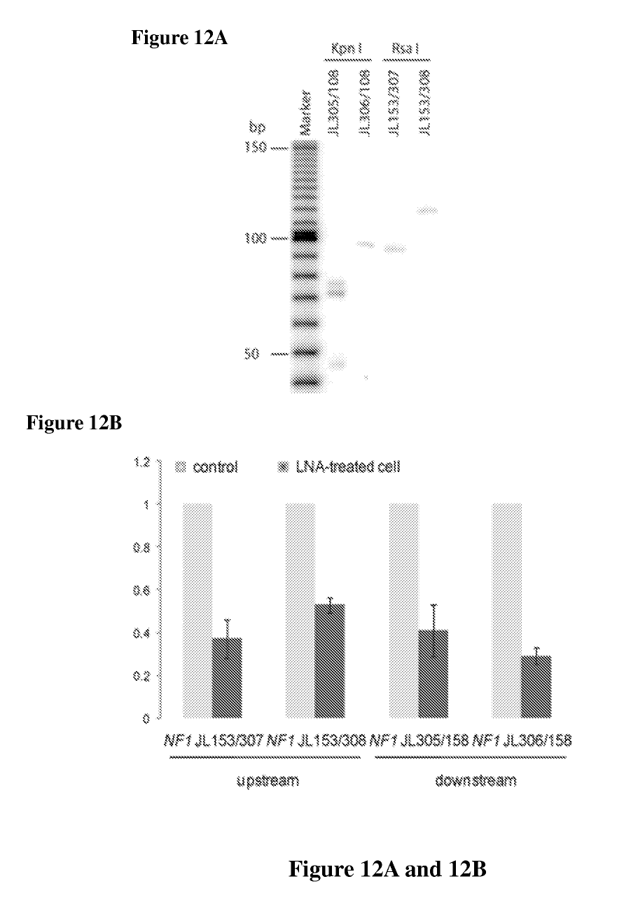

SNPs were assessed in 6 fibroblast cell lines at the NF1 locus in order to distinguish the transcripts from the two alleles, and one cell line GM01859, was found to have informative SNPs rs1801052 and rs2040793, which could be used to determine if linked LNA targeted both of the NF1 alleles. linked LNA targeting to the ECR15 region was achieved by the use of two LNA oligonucleotides with 20-mer single strand regions (LNA1 and LNA2) that bind to genomic DNA sense and antisense strands of ECR15, respectively; two 7-mer linker that are complementary to each other connect the two single strand LNA (FIG. 3B-C). Allele-specific PCR primers were also designed. The RT-PCR products were obtained using each pair of primers followed by restriction enzyme digestion by Rsa I for SNP rs1801052 and Kpn I for SNP rs204079; only one allele was amplified at a time (FIG. 12A). This finding demonstrated that the allele-specific primer pairs could be used in real time-PCR to quantify allele-specific expression of NF1 in linked LNA treated GM01859 cells.

Linked LNA was introduced into cells by microporation with high efficiency (63%), as indicated by the co-transfected pEGFP. NF1 gene expression was determined by real time-PCR using allele-specific PCR primers pairs. In the linked LNA-treated cells, expression from each NF1 allele decreased significantly (FIG. 12B).

FIGS. 12A and 12B thus show altered NF1 expression in linked LNA-treated GM01859 cells. (12a). Allele-specific RT-PCR of NF1 upstream 5′-end and downstream 3′-end transcripts of ECR15. RT-PCR products were subjected to Kpn I or Rsa I digestion, respectively. Each primer pair for RT-PCR was labeled above its corresponding lane. The results demonstrate the ability of each primer pair to specifically amplify its allele-specific transcript of NF1 in RT-PCR. (12b). Quantification of allele-specific expression of upstream 5′-end and downstream 3′-end transcripts of ECR15NF1 by real time-PCR. Allele-specific primer pairs were used in each reaction shown under their corresponding columns. Two repeat experiments were performed in quadruplicate for each allele-specific real time-PCR. Values are mean±SD from the two assays. Light gray column: normal culture cells; dark gray column: linked LNA-treated cells.

### Linked LNA Binding to ECR15 Changes Protein-DNA Interactions

Local DNA conformation change at ECR15 was assessed using electrophoresis in a 2% agarose gel. The linked LNA-bound ECR 15 DNA fragment amplified by PCR appeared as a broad band, while the ECR15 DNA fragment itself migrated as a thin, sharp band. We hypothesize that the broad gel pattern of the linked LNA-bound ECR15 DNA fragment was caused by linked LNA-induced DNA conformation changes, affecting its electrophoretic behavior. Twenty randomly selected clones from both linked LNA treated and untreated cells were sequenced, and there was no DNA content change in the linked LNA-targeted ECR15 region in any of the clones.

We then determined if linked LNA binding at the ECR15 CTCF binding site affects CTCF and RNA Pol II binding at this ECR region. Linked LNA binding enhanced CTCF binding by ˜25% at ECR15. CTCF binding decreased by >50% at the ECR4 region in linked LNA-treated cells (FIG. 13A), a region not targeted by the linked LNA. There was no significant change in RNA Pol II binding at ECR15 or ECR4 in the linked LNA treated GM01859. However, linked LNA treatment increased RNA Pol II binding by ˜25% at ECR11, but there was no change in CTCF binding at that region (FIG. 13B). No significant change in CTCF or RNA Pol II binding at ECR17, ECR18 and ECR20 site were detected by ChIP assay in the linked LNA-treated cells. A CTCF-ChIP assay at CTCF binding site 3 of the H19 imprinting control region (ICR) served as a control in the experiment; linked LNA treatment did not alter CTCF binding at that site (FIG. 13C). These data demonstrate the target specificity of the linked LNA. Linked LNA altered CTCF and RNA Pol II binding at their target sites at ECR15 specifically, but there was no change in their binding at a region not targeted by the linked LNA, such as ECR17, ECR18, ECR20 and CTCF binding site 3 of the H19 ICR. However, the mechanism by which CTCF binding decreased at the ECR4 site is not clear.

### Linked LNA Binding to ECR15 Alters Long-Range DNA Interactions at the NF1 Locus

Since a specific linked LNA targeted to ECR15 altered CTCF and RNA Pol II binding at the ECR15, ECR4 and ECR11 regions of the NF1 gene, we suspected that it might also alter long-range interactions at the NF1 locus. ECR4 and ECR11 are 158 kb and 30 kb away from ECR15, respectively. Primers were designed at Hind III sites near ECR4, ECR11 and ECR15. We used the chromatin conformation capture (3C) assay to identify chromatin interactions among these DNA regions. In linked LNA-treated cells, an ECR15/ECR4 DNA fragment was detected, but no ECR15/ECR11 fragment was found. However, the ECR15/ECR11 fragment was detected in normal cells, but no ECR15/ECR4 fragment was observed (FIG. 3A), indicating that linked LNA binding to the ECR15 region alters DNA long-range interactions at the NF1 locus. No DNA interaction between ECR15 and ECR17, ECR18 and ECR20 was detected in either linked LNA-treated or normal cells in this study.

To explore how the ECR15/ECR4 interaction could lead to the down-regulation of NF1 expression, the ECR15 region was cloned into pGL4.23 [luc2/minP] and expressed in HeLa cells. Compared with control cells that had been transfected with an empty vector, firefly luciferase activity decreased by >70% in the pGL4.23-ECR15-transfected cells (FIG. 14B). This result suggests that the ECR 15 region may act as a silencer at the NF1 locus. Linked LNA binding to ECR15 induced the establishment of an ECR15/ECR14 long-range DNA interaction in the linked LNA-treated cells (FIG. 14), juxtaposing the ECR15 silencer close to the promoter region of the NF1 gene and suppressing its expression. The ECR15 silencer is normally distant from the NF1 promoter region, as it tied up in an ECR15/ECR11 DNA long-range interaction that is not close to the promoter (FIG. 2).

### CONCLUSION

The above specific description is meant to exemplify and illustrate the invention and should not be seen as limiting the scope of the invention, which is defined by the literal and equivalent scope of the appended claims. Any patents or publications mentioned in this specification are intended to convey details of methods and materials useful in carrying out certain aspects of the invention which may not be explicitly set out but which would be understood by workers in the field. Such patents or publications are hereby incorporated by reference to the same extent as if each was specifically and individually incorporated by reference and contained herein, as needed for the purpose of describing and enabling the method or material referred to.

1. A method for inserting a DNA construct into a target DNA in a cell, the target DNA having a coding strand and a template strand, said method comprising the steps of providing in the cell:
(a) locked nucleic acid molecules comprising (i) a sequence complementary to a portion of the target DNA on a template strand, (ii) a sequence complementary to a portion of the target DNA on a coding strand, and (ii) a sequence complementary to a locked nucleic acid molecule, whereby the locked nucleic acid molecules bind to both the template strand and the coding strand of the target DNA;
(b) a vector comprising the DNA construct and further comprising adjacent sequences complementary to sequences in the locked nucleic acid molecules, whereby the locked nucleic acid molecules bind to vector; and
(c) a recombinase construct which provides a protein which acts to combine the vector DNA and the target DNA, thereby causing insertion of the DNA construct into the target DNA sequence in the cell.

2. A method according to claim 1, wherein the DNA construct comprises a selectable marker.

3. The method of claim 2 wherein the selectable marker is an antibiotic resistance gene, and the DNA construct further comprises a promoter for expressing the marker.

4. The method of claim 1 wherein the DNA construct comprises a detectable marker.

5. The method of claim 4 wherein the detectable marker is a fluorescent protein.

6. The method of claim 1 wherein the cell is eukaryotic and further may be selected from the group consisting of mammalian and human.

7. The method of claim 1 further comprising the step of providing in the cell locked nucleic acid molecules which comprise a pair of linked locked nucleic acids.

8. The method of claim 1 wherein the step of providing in the cell a recombinase comprises introducing into the cell an expression vector expressing a specific recombinase.

9. The method of claim 8 wherein the recombinase is Rec A.

10. The method of claim 1 wherein the locked nucleic acid molecules have sequences complementary to at least a portion of the target DNA sequence, the locked nucleic acid sequences being at least about 18 nucleotides in length.

11. The method of claim 1 wherein the construct to be inserted blocks gene transcription.

12. The method of claim 1 wherein the construct to be inserted alters gene transcription.

13. The method of claim 1 wherein the target sequence is a CCCTC-binding factor (CTCF) binding site.

14. The method of claim 1 wherein locked nucleic acid molecules and vectors are applied to more than one gene simultaneously.

15. A kit for modifying expression of a selected gene in a genome of a cell by altering a target region of a DNA sequence of the selected gene, comprising:
(a) locked nucleic acid molecules having sequences complementary to at least a portion of the target region of the DNA sequence on opposite template and coding strands, to form displaced strands; and
(b) a vector comprising the DNA construct to be inserted and further comprising sequences complementary to the locked nucleic acids, whereby the locked nucleic acids bind to both the vector and the target region.

16. The kit of claim 15 further comprising a vector expressing a recombinase which combines the vector and the DNA sequence, thereby causing insertion of the DNA construct at the target region of DNA sequence in the cell.

17. The kit of claim 15 further comprising vectors and locked nucleic acid molecules for a plurality of different genes.

18. A method for suppressing gene expression of a selected gene in a genome of a cell, comprising the step of binding a pair of linked locked nucleic acids to a specific sequence that is a CCCTC-binding factor (CTCF) binding site that affects chromatin organization in the genome.

19. The method of claim 18 wherein the gene is human.

20. The method of claim 18 wherein the gene is a neurofibromatosis gene.

21. The method of claim 18 further comprising the step of identifying the binding site using chromatin immunoprecipitation.

22. The method of claim 18 further comprising the step of identifying a CTCF binding site using a computerized CTCF binding site prediction tool and selecting a site within the locus of the gene and that has a unique sequence within the genome.
